(19) United States
(12) Patent Application Publication
(10) Pub. No.: US 2005/0142067 A1
(43) Pub. Date: Jun. 30, 2005

(54) Zintrodes, multitrodes and uses thereof

(76) Inventors: Christopher Frederickson, Galveston, TX (US); Carol Fierke, Ann Arbor, MI (US); Richard Thompson, Baltimore, MD (US)

Correspondence Address: Benjamin Aaron Adler;ADLER & ASSOCIATES, 8011 Candle Lane, Houston, TX, 77071, US

(21) Appl. No.: 10/929,924

(22) Filed: Aug. 30, 2004

Related U.S. Application Data
(60) Provisional application No. 60/499,175, filed on Aug. 29, 2003.

(51) Int. Cl. A61B 5/00; C12Q 1/00

(52) U.S. Cl. 424/9.6; 205/777.5

(57) ABSTRACT

Provided herein are zintrodes to measure intracranial free zinc levels in an individual or free zinc levels in solution. The zintrodes and multitrodes comprise a zinc chelator and a fluorophore or may comprise only a zinc-chelating fluorophore, an optical fiber having an optical tip, and a means of entrapping the zinc chelator and the fluorophore or the zinc-chelating fluorophore proximate to the optical tip within the zintrode. The zintrode may comprise a multitrode. Also provided are methods of real-time buffering of zinc ion levels in vivo in brain tissue to treat an excitotoxic neural injury using the zintrodes and multitrodes described herein. Additionally, provided herein are a system and method to measure pZn in solution.

### CROSS-REFERENCE TO RELATED APPLICATIONS

This nonprovisional application claims benefit of provisional U.S. Ser. No. 60/499,175, filed Aug. 29, 2003, now abandoned.

### BACKGROUND OF THE INVENTION

1. Field of the Invention

The present invention relates generally to the fields of biosensors, neurology, diagnostic medicine and therapeutics. More specifically, the present invention relates to the use of zinc-sensing zintrodes, i.e., zintrodes, or multitrodes for analyzing, monitoring and/or buffering of free zinc levels.

2. Description of the Related Art

Zinc, like the amino acid glutamate, has two distinct roles in the central nervous system: structural and signaling. Zinc, as is glutamate, is an integral component of thousands of different proteins, none of which are functional if the zinc is omitted or removed (1-3). The zinc metalloproteins include transcription factors containing zinc fingers, structural proteins and enzymes from all six enzyme classes, and are present in virtually every cellular organelle of every cell type in the brain, the body, and, in fact, in the biosphere (4). Not surprisingly, zinc deficiency is devastating (1) and, in the prevailing rat model of complete zinc-free malnutrition, is fatal within two weeks (5).

In addition to its role in proteins, zinc is vital as the “free” signal ion, which is stored in the pre-synaptic vesicles of specific cerebrocortical neurons, and released from them in an impulse and calcium-dependent manner (6). First discovered in the mid 1980's the synaptically-released zinc signals are now recognized as a vital part of cerebrocortical function and pathology (7-10). The membrane spanning pump responsible for pumping zinc into the pre-synaptic vesicles has been identified (11-12) and the kinetics and biophysics of zinc release are gradually being characterized (13-14), as is the global neuroanatomy of the zinc-containing neuronal architecture of the cerebral cortex (15-17).

Zinc-containing pathways virtually all originate in the cerebral cortex, where they are concentrated in both the “limbic” (allo) and “neo” (iso) cortical regions. The amygdala, hippocampal formation and perirhinal cortex are the major points of origin for these pathways and zinc-containing pathways that originate from the limbic system reach virtually all limbic and isocortical regions (4, 7, 16-18). Moreover, corticocortical association fibers, including commissural fibers, are predominantly of the zinc-containing variety.

The cortical zinc-containing neurons are a subset of the cortical glutamatergic neurons. All cortical zinc-containing neurons are glutamatergic neurons, although not all glutamatergic neurons are zinc-containing neurons (17, 19). Combining the anatomy and the pharmacology, it is not surprising that synaptically-released zinc is intimately involved in glutamatergic excitatory signaling in the cerebral cortex. Both the tonic regulation of cortical excitability (20) and the phasic driving of the glutamatergic synaptic plasticity (LTP) are crucially dependent upon synaptic zinc signals (13-14, 21-22).

While zinc is thus essential for health, it has also been implicated in the neuropathology of disease states such as Alzheimer's disease (23-24), epileptic seizures (25-26) and cerebral ischemia and traumatic brain injury (27-30). Recent studies have shown that both oxidative and nitrosylative stresses can liberate zinc from metalloproteins in vivo (31).

Zinc is thus emerging as a key therapeutic target for disorders involving the exitatory synapse, as zinc is a key factor in both epileptic disorders and the whole realm of pathologies which hinge upon the so-called excitotoxic cascade. Myriad evidence from zinc-addition paradigms, zinc chelation and specific zinc-altering genetic knockout and transgenic mice, support the conclusion that synaptically-released zinc is a tonic, anti-convulsive down-regulator of EAA (Essential Amino Acids) function in the cerebral cortex (25, 32-36). The possible role of zinc in modulating GABA synapses after kindled-sprouting has also been suggested (37), although the effects of zinc chelation on such kindling remain to be explored.

It is clear that zinc is directly involved in the final common pathway of neuronal injury. Evidence has been adduced that zinc excitotoxicity involves a release of excess zinc ions from synaptic terminals. For example research has shown that traumatic brain injury liberates zinc from axonal boutons with the excitotoxic zinc flood then traversing the synaptic cleft into post-synaptic neurons, causing apoptosis (29). In vitro studies have demonstrated that cultured neurons exposed to 300-500 nM Zn⁺² for 5 minutes develop widespread degeneration over a 24 hour period (38) and that, following brief exposure to 400 μM zinc, cortical cells exhibited DNA fragmentation, increased poly(ADP-ribosyl)ation and decreased levels of NAD and ATP and subsequently underwent cell death (39).

Although zinc levels in excess of 300 nM have been shown in vitro to cause neuronal death, neither the in vivo upper threshhold level for free zinc above which such neurotoxic effects occur, nor the in vivo lower threshhold level of free zinc, below which seizures will occur, nor the in vivo basal Zn⁺² level in the brain, has heretofore been determined. For research on zinc signaling to grow and mature, research tools are required to allow in vivo measurement of low levels of physiological zinc in fluids such as serum and CSF.

In animal models of ischemia, seizures or head trauma, chelation of free zinc has prevented up to 80% of neuronal loss in trauma and stroke (33-34, 40) and specific chelation of cerebral zinc has reversed both the plaque and cognitive defects of Alzheimer's disease (41). The use of chelation therapy is thus the subject of significant medical investigation. However, since chelating zinc to too low a level can induce seizures (25, 32) and can facilitate apoptosis (42) there is a critical need to be able to measure and monitor free zinc levels in the brain to characterize and control chelation therapies.

An zintrode is a chemical sensor/probe based on an optical fiber(s) which is used to transmit excitation light from an illumination source to an analyte captured within an area of the probe, and to collect the return signal, either emission or scattering, onto a detector. The fiber optic probe is small and enables in vitro or in vivo real-time monitoring of biological fluids and in vivo implantation. Typically, the zintrode body is made of an inert, biocompatible material, e.g. a fluorocarbon, with a port or an area in the wall that is fitted with a material permeable to the analyte, e.g. Zn⁺², which reacts with a reagent contained within the body of the sensor (U.S. Pat. No. 5,116,759).

The inventors have recognized a need for improvement in detection technology for determining in vivo either the basal range of zinc ions in the brain or the lower and upper threshholds for deleterious effects. These ranges are required in order to facilitate buffering within that “safe” range during therapeutic treatment such as chelation to reduce zinc excitotoxicity caused by traumatic brain injury, ischemia or stroke or zinc supplementation to reduce seizures. The small size and real-time analysis capability of an zintrode makes it ideal for in vivo monitoring of a chemical species in response to a therapeutic treatment, for which that species must be controlled within a specific range, e.g. buffering. This is required to achieve therapeutic efficacy and to avoid deleterious effects due to either too little or too much of the chemical species.

The prior art is deficient in the lack of in vivo monitoring methods of a chemical species to maintain physiological or therapeutic levels of the chemical species in an individual. Specifically, the prior art is deficient in devices and methods to monitor in vivo zinc levels in an individual in real-time. The present invention fulfills this long-standing need and desire in the art.

### SUMMARY OF THE INVENTION

The present invention is directed to, inter alia, a zintrode to measure free zinc levels in a biological solution. The zintrode comprises a chelator to chelate free zinc ions, a fluorophore, an optical fiber comprising an optical tip, and means of entrapping the chelator and the fluorophore proximate to the optical tip within the zintrode. The zintrode and a zinc calibration solution comprise a kit.

The present invention also is directed to a method of real-time buffering of zinc ion levels in vivo in brain tissue to treat a pathological condition characterized by abnormal levels of zinc. The zintrode described herein is implanted intracranially into the brain tissue and is used to measure the level of zinc ions in the brain tissue. A pharmacologically effective dose of an agent is administered to remove an amount of zinc ions sufficient to maintain the level of remaining zinc ions within a physiologically acceptable range. The zinc ion level is continuously monitored and if the monitored level of zinc ions increases above the maintenance level the agent is readministered. This achieves real-time buffering of zinc ion levels in brain tissue which treats the pathological condition.

The present invention is directed further to an implantable multitrode to measure intracranial physiological parameters in brain tissue in an individual. The multitrode comprises an intra-arterial tritrode having a fiber optic channels to monitor one of blood pH, pO₂ or pCO₂ parameters, an implantable zintrode to measure free zinc ion levels in the brain tissue and a means of electronically monitoring the multitrode. The implantable zintrode comprises a zinc-chelating fluorophore, an optical fiber having an optical tip; and means of attaching the zinc-chelating fluorophore to the optical tip.

The present invention is directed further to a related method of real-time buffering of zinc ion levels in vivo in brain tissue to treat a pathological condition characterized by abnormal levels of zinc using the multitrode described herein. The multitrode described herein is implanted into the brain tissue. The tritode comprising the multitrode continuously monitors blood pH, pO2 and pCO2. The zintrode comprising the multitrode measures and monitors zinc ion levels in brain tissue as described supra.

The present invention is directed further still to a system to measure pZn in a solution. The system comprises a zintrode, a means for holding the solution and optical components to measure fluorescence. The zintrode comprises a zinc-chelating fluorophore, an optical fiber comprising an optical tip and a means of entrapping said zinc-chelating fluorophore proximate to the optical tip within the zintrode. Optionally, the system may comprise a means for removing zinc-binding ligands from the solution prior to measuring pZn.

The present invention is directed further still to a method of measuring pZn in a solution. The solution is contacted with a zintrode in the system described herein. Free zinc ions in the solution diffuse into the zintrode for a period of time. An excitation maximum wavelength is delivered to the zinc-chelating fluorophore in the zintrode via the optical tip comprising said zintrode and an emission maximum wavelength emitted by the zinc-chelating fluorophore is measured via the optical components comprising the system. The concentration of free zinc ions correlates with the emission maximum wavelength thereby measuring the pZn of the solution. Optionally, prior to contacting the solution with the zintrode, the solution may be placed into a 100 MW dialysis tube contained within a cuvette and dialyzed against a buffer to remove zinc-binding ligands from the solution.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1A depicts a calibration curve for a CA-ABDN zinc measuring system. The concentration of apoCA was 50 μM, so the 50% occupancy point for the holoCA+ABDN in this stoichiometric titration is at 25 μM.

FIG. 1B depicts the abrupt rise in the free zinc concentration in the dialysate during perfusion of the ventricle in two rats. A total of four hours is shown.

FIG. 1C depicts the baseline pZn in the anesthetized rat brain.

FIG. 1D depicts free zinc in human cerebral spinal fluid samples using the apoCA dialysis method.

FIG. 2A depicts the ischemia-induced release of zinc for one of more than a dozen rabbits studied.

FIG. 2B depicts the NO* infusion released zinc into microdialysis fluid in acutely prepared rabbits.

FIG. 3 depicts fiber-optic monitoring of free zinc in the dog during ischemia.

FIGS. 4A-4B demonstrate the permability characteristics of the microdialysis membrane for DPSA and free zinc. FIG. 4A shows that DPSA does not leak out of the dialysis chamber even after 24 hours. The lower line is the fluorescence spectrum of the HEPES external medium and the upper line is the fluorescence spectrum of the microdialysis chamber containing DPSA. FIG. 4B shows that zinc can pass through the microdialysis membrane. The blue line is the fluorescence spectrum of the microdialysis chamber containing apoCA and DPSA before it is exposed to an external medium containing free zinc and the lower line is the fluorescence spectrum of that microdialysis chamber after exposure to an external medium containing 50 μM HEPES and 10 μM zinc.

FIG. 5 depicts the dialysis recovery function for the apoCA-DNSA zinc measuring system. The left curve shows values obtained when zinc calibration standards were measured directly with the apoCA-DNSA system, and the right-shifted curve shows values obtained when the same zinc calibration standards were sampled by microdialysis, and the dialysates were then measured.

FIG. 6 shows the fluorescence response of a fiber optic biosensor to concentration of free zinc in solution. The upper inset shows the biosensor construction, in which the CA is tethered to a fluorophore (Oregon Green) and the tethered CA-reporter pair is embedded in a sol-gel matrix which is polymerized onto the end of the optical fiber. The sol-gel pores trap the CA-reporter conjugate, while allowing free zinc ions to pass freely. The lower inset is a photo-micrograph of the actual fiber tip.

FIG. 7 demonstrates that dansylamide tethered to controlled-pore-glass (CPG) beads with a 12-carbon linker has a robust fluorescent response to 360 nm excitation.

FIGS. 8A-8B depict the component parts (FIG. 8A) and assembly of the components (FIG. 8B) of a zinc sensing zintrode.

FIG. 9 depicts a schematic diagram showing the component parts and side views of the assembly of an alternate zinc-sensing zintrode.

FIG. 10 is a schematic diagram of an optical fiber tip bent to form a U shape and having a laser-cut notch used in another zinc-sensing zintrode.

FIG. 11 depicts a microdialysis tube holder assembly, the zinc sensing zintrode of FIG. 9, an excitation source and two photodiodes with bandpass filters for a DNSA reporter.

FIGS. 12A-12C depict methods of entrapping a zinpyr or rhodafluor fluorescent dye within an zintrode.

FIG. 13 demonstrates that glass-tethered small molecules detect free zinc. The fluorescent zincprobe, ZP1 (Em 530) is attached to a microscope slide by a polymeric matrix. A large drop of the ZP1 polymer was cured onto a microscope slide (tan oval) and small drops of either HEPES (left drop) or HEPES+ZnSO4 (right drop) were placed on the surface. The upper panel shows the resulting images in the fluorescent microscope (4×). Washing with HEPES extinguished the fluorescence. Four replications were done (not shown). The lower panel shows a similar experiment with the fluorescent probe TFLZn (Em=505). However, the intensity change was too bright, i.e., ˜15-fold to capture at fixed camera setting, so the result is shown schematically, with the fluorescent image at right shown at a different exposures.

FIGS. 14A-14C demonstrate that ZP1 attached to glass substrate responds to free zinc. FIG. 14A is a schematic depicts a spin-coat of ZP1 in polymer attached to the inside of a quartz tube through which zinc-containing fluids flow. FIG. 14B is a spectra showing reversibility, with water in the tube before zinc (black), zinc in the tube (50 μM; blue), then water again (red). FIG. 14C shows sequential measurements at 530 nm (l/sec) as first water, then zinc (50 μM), were flowed into the tube. It is inferred that the major bolus of the zinc arrived at the portion of the tube being monitored at approximately second # 8, meaning that the response spanned about 8-12 seconds.

FIG. 15 demonstrates the efficacy of the pZn Meter in cell biology. ZnCl₂ was added in the concentrations shown on the abscissa and the resulting concentrations of free zinc (pZn) were measured. Note that the pZn hardly changed, even though up to 90 μM was added. After obtaining the proprietary formula for the medium from the manufacturer, a multi-ligand model was made using MineqL and the expected free zinc was calculated.

FIGS. 16A-16B depict calibration curves for the pZn Meter. FIG. 16A shows the curve using metal buffer to control free zinc. The [ZP1] is <<than the effective [Zn2+ free], because the metal buffer (50 μM bicine) can supply a large total number of Zn ions, while keeping the concentration of zinc ions fixed. Thus, in this calibration, the fluorescence intensity midpoint is approximately equal to the KD, ˜0.5 nM].[Zp1]=100 nM. Note too, that the fluorescence intensity continues to rise to the right, because that portion of the curve is in the stoichiometric regime of the titration. This curve would plateau at [Zn]={ZP1}=100 nM. FIG. 16B shows the calibration curve using a ZP1 concentration of 20 nM of zinc (as ZnSO4). Because no metal buffer is used and because the ZP1 concentration is much higher that the zinc concentration, this is a stoichiometric, or “percent occupancy” titration, in which the fluorescence intensity is simply proportional to the molar ratio,[Zn]:[ZP1].

FIG. 17 depicts the clean-up cuvette for the pZn meter.

### DETAILED DESCRIPTION OF THE INVENTION

In one embodiment of the present invention there is provided a zintrode to measure free zinc levels in a solution, comprising a chelator to chelate free zinc ions; a fluorophore; an optical fiber comprising an optical tip; and means of entrapping the chelator and the fluorophore proximate to the optical tip within the zintrode. In all aspects of this embodiment the solution may be a growth media comprising fetal calf serum, cerebral spinal fluid, blood, seminal serum, saliva, tears, urine, or synthetic salt solutions.

In one aspect of this embodiment the chelator is a wild-type apoenzyme or genetically engineered mutant thereof. Examples of the apoenzyme are wild-type apo-carbonic anhydrase, a S166C mutant, a H36C mutant or a E117A mutant. Also, in this aspect the fluorophore may be DPSA, DNSA, Butyl-DPSA or polyABDN. Further to this aspect the fluorophore may be covalently tethered to controlled-pore glass beads or to a protein or physically bound to a polyethylene thread.

Also in this aspect the means of entrapping may be a microdialysis chamber comprising a semi-permeable membrane positioned over the tip of the optical fiber and attached thereto such that the apoenzyme and the fluorophore are entrapped therein. Alternatively, the means of entrapping may be a water-soluble polymer matrix polymerized onto the tip of the optical fiber where the fluorophore is conjugated to the apoenzyme and embedded therein. An example of a water-soluble polymer matrix is a sol-gel, a hydrogel or a microgel.

In another aspect of this embodiment the zinc chelator is the fluorophore. The zinc-chelating fluorophore may be a zinc-chelating fluorescent dye. Examples of a zinc-chelating fluorescent dye are a zinpyr dye, TSQ or a congener thereof or a rhodafluor dye. The zinpyr dye may be 9-(O-carboxyphenyl)-2,7-dichloro-4,5-bis[bis(2-pyridylmethyl)-aminomethyl]-6-hydroxy-3-xanthanone, 9-(O-carboxyphenyl)-4,5-bis[bis(2-pyridylmethyl)-aminomethyl]-6-hydroxy-3-xanthanone, 9-(O-carboxyphenyl)-2-chloro-5-[2-{bis(2-pyridylmethyl)aminomethyl}-N-methylaniline]-6-hydroxy-3-xanthanone. The zinpyr dye also may be ZP-3 or ZPN. The congener of TSQ may be TFLZN. An example of a rhodafluor dye is (1-[9′-(O-carboxyphenyl)-6′-amino-2′-chloro-3′-xanthanone]-4,10-(diethyl)-7-(2-pyridyl methyl)-1,4,7,10-tetraazacyclododecane.

Further to this aspect the means of entrapping may be a covalent bond between the zinc-chelating fluorophore and the optical tip. Alternatively, the means of entrapping may be a water-soluble polymer matrix with the zinc-chelating fluorophore embedded therein such that the polymer matrix is polymerized onto the tip of the optical fiber. The water-soluble polymer matrix is as described supra.

Still further to this aspect the means of entrapping may be a microdialysis chamber comprising a capillary tube containing the zinc-chelating fluorophore at one end and the optical tip inserted therein. A semi-permeable membrane is positioned over the capillary tube and attached thereto such that the zinc-chelating fluorophore is entrapped therein. In a related aspect the capillary tube may contain the zinc-chelating fluorophore at one end in a solution of a highly viscous low polarity liquid to entrap the zinc-chelating fluorophore entrapped therein. The end of the capillary tube may be narrowed to 50 microns. The highly viscous low polarity liquid may be a high molecular weight alcohol, a high molecular weight fatty acid or a silicone oil.

In a related embodiment, there is provided a kit comprising the zintrode described supra and a zinc calibration solution.

In a related embodiment of the present invention there is provided a method of real-time buffering of zinc ion levels in vivo in brain tissue to treat a pathological condition characterized by abnormal levels of zinc, comprising the steps of a) intracranially implanting the zintrode described supra into the brain tissue; b) measuring the level of zinc ions in the brain tissue with the zintrode; c) administering a pharmacologically effective dose of an agent to remove an amount of zinc ions sufficient to maintain the level of remaining zinc ions within a physiologically acceptable range; d) continuously monitoring the zinc ion level in step b); and e) repeating step c) if the monitored level of zinc ions increases above the maintenance level thereby buffering the zinc ion levels in brain tissue in real-time to treat the pathological condition.

Further to this embodiment the method comprises implanting an intra-arterial tritrode comprising fiber optic channels to measure one of blood pH, pO₂ or pCO₂, measuring a level of blood pH, pO₂ or pCO₂ and monitoring said blood pH, pO₂ or pCO₂ levels during buffering of zinc ion levels. Also further to this embodiment, the zinc ion level is measured by diffusing zinc ions into the zintrode for a period of time; delivering an excitation maximum wavelength to a fluorophore in the zintrode via an optical tip comprising the zintrode; measuring emission maximum wavelengths where the fluorophore emitts one emission maximum wavelength or two different emission maximum wavelengths; and correlating the level of zinc ions with said one emission maximum wavelength or ratiometrically from said two different emission maximum wavelengths.

In one aspect of this further embodiment the fluorophore complexes with a wildtype or mutated apocarbonic anhydrase and holocarbonic anhydrase where each complex has a different emission maximum wavelength. The wild-type and mutated carbonic anhydrases and the fluorophores binding thereto are as described supra. In another aspect fluorophore complexes with said zinc ions where the zinc ion level is determined from the emission maximum wavelength of the fluorophore-zinc complex. The fluorophore may be a zinpyr dye as described supra, TSQ or TFLZN. In yet another aspect the fluorophore complexes with said zinc ions where the zinc ion level is determined ratiometrically from the emission maximum wavelengths of the fluorophore and the fluorophore-zinc complex.

Further still to this embodiment the zinc ion levels are continuously monitored via a base unit comprising a high resolution graphic display screen and a sensor interface unit. The sensor interface unit comprises a light source, photodiode detectors, optionally with bandpass filters, and electronic data amplification elements.

In all aspects of this embodiment the physiologically acceptable range of zinc ions is about 0.1 nM to about 20 nM. Preferably, the range may be about 0.5 nM to about 5.0 nM. More, preferably the range is about 1 nM. Also, in all aspects the agent to remove zinc ions from the brain tissue is a zinc-release blocker, a zinc chelator or a zinc channel blocker. An example of a zinc chelating agent is clioquinol. Furthermore, in all aspects the pathological condition may be an excitotoxic neural injury, epilectic seizure, ischemia, Alzheimer's disease or traumatic brain injury.

In another embodiment of the present invention there is provided an implantable multitrode to measure intracranial physiological parameters in brain tissue in an individual, comprising an intra-arterial tritrode comprising fiber optic channels to monitor one of blood pH, pO₂ or pCO₂ parameters; an implantable zintrode to measure free zinc ion levels in the brain tissue which comprises a zinc-chelating fluorophore; an optical fiber having an optical tip; and means of attaching said zinc-chelating fluorophore to the optical tip; and means of electronically monitoring the multitrode.

In this embodiment the zinc-chelating fluorophore is a zinc-chelating fluorescent dye. These fluorescent dyes may be a zinpyr dye or TSQ or a congener thereof, as described supra. Also in this embodiment the means of attaching the zinc-chelating fluorophore to the optical tip may be covalent attachment or embedding the zinc-chelating fluorophore into a water soluble polymer matrix attached to the optical tip as described supra. Furthermore, in this embodiment the means of electronically monitoring the multitrode is a base unit that comprises a high resolution graphic display screen and a sensor interface unit. The sensor comprises a light source, detection components for the tritrode, photodiode detectors, optionally with bandpass filters, for said zintrode, and electronic data amplification elements.

In a related embodiment there is provided a method of real-time buffering of zinc ion levels in vivo in brain tissue to treat a pathological condition characterized by abnormal levels of zinc, comprising the steps of a) implanting the multitrode described supra into the brain tissue; b) continuously monitoring blood pH, pO2 and pCO2 via the tritrode comprising the multitrode; c) measuring the level of zinc ions in the brain tissue with the zintrode comprising the multitrode; d) administering a pharmacologically effective dose of an agent to remove an amount of zinc ions sufficient to maintain the level of remaining zinc ions within a physiologically acceptable range; e) continuously monitoring the zinc ion level in step c); and f) repeating step d) if the monitored level of zinc ions increases above the maintenance level thereby buffering the zinc ion levels in brain tissue in real-time to treat said pathological condition.

Further to this embodiment the zinc ion level in brain tissue is measured by diffusing zinc ions into the zintrode for a period of time; delivering an excitation maximum wavelength to the zinc-chelating fluorophore in the zintrode via an optical tip comprising the zintrode; measuring an emission maximum wavelength emitted by the zinc-chelating fluorophore; and correlating the level of zinc ions with the emission maximum wavelength. The zintrode and zinc-chelating fluorophores are as described supra for the multitrode.

In aspects of this embodiment the tritrode may be implanted via an arterial introducer or via a cannula. In another aspect the zintrode may be implanted via a cannula. In all aspects of this embodiment the physiological ranges, the zinc-removing agent and the pathophysiological condition are as described supra.

In yet another embodiment of the present invention there is provided a system to measure pZn in a solution, comprising a zintrode which comprises a zinc-chelating fluorophore; an optical fiber comprising an optical tip; and means of entrapping the zinc-chelating fluorophore proximate to the optical tip within the zintrode; means for holding the solution; and optical components to measure fluorescence. Further to this embodiment the method comprises a means for removing zinc-binding ligands from the solution prior to measuring pZn. The removing means may comprise a 100 MW dialysis tube; a cuvette containing said dialysis tube; and a buffer.

In all aspects of this embodiment the zinc-chelating fluorophore is a zinc-chelating fluorescent dye. These fluorescent dyes may be a zinpyr dye or TSQ or a congener thereof, as described supra. Also in all aspects of this embodiment the solution may be a growth media comprising fetal calf serum, cerebral spinal fluid, blood, seminal serum, saliva, tears, urine, or synthetic salt solutions.

In one aspect of this embodiment the means for holding the solution is a cuvette. In another aspect of this embodiment the means of entrapping the zinc-chelating fluorophore may be a covalent bond with the optical tip of the optical fiber. In a further aspect the means of entrapping may be a water-soluble polymer matrix with the zinc-chelating fluorophore embedded therein. In one example of such entrapping, the polymer matrix is polymerized onto the optical tip. Alternatively, the polymer matrix may be polymerized onto the inner surfaces of a cuvette used as the means for holding the solution. The water-soluble polymer matrix is as described supra.

In a related embodiment there is provided a method of measuring pZn in a solution, comprising a) contacting the solution with a zintrode comprising the system described supra; b) diffusing free zinc ions in the solution into the zintrode for a period of time; c) delivering an excitation maximum wavelength to the zinc-chelating fluorophore in said zintrode via the optical tip comprising the zintrode; d) measuring an emission maximum wavelength emitted by the zinc-chelating fluorophore via the optical components comprising the system; and e) correlating the concentration of free zinc ions with the emission maximum wavelength thereby measuring the pZn of the solution. Further to this embodiment the method may comprise, prior to step a), placing the solution into a 100 MW dialysis tube contained within a cuvette and dialyzing the solution against a buffer to remove zinc-binding ligands.

In one aspect of this embodiment the concentration of free zinc ions may be greater than about 50 picomolar. The measurable pZn may be less than about 10.3. In all aspects of this embodiment the zinc-chelating fluorophore is as described supra.

The following terms shall be interpreted according to the definitions set forth below. Terms not defined infra shall be interpreted according to the ordinary and standard usage in the art.

As used herein, “pZn” shall refer to the concentration of rapidly-available zinc ions.

As used herein, “zintrode” shall refer to a miniaturized system allowing optical detection of an analyte, based on a fiber optic probe via which excitation of an analyte or analyte complex and collection of a signal, e.g., luminescence, fluorescence or scattering, from the excited analyte or analyte complex can be achieved.

As used herein “zintrode” shall refer to a zinc-sensing zintrode.

As used herein, “multitrode” shall refer to a device combining multiple zintrodes and/or electrodes for measurement of physiological parameters.

As used herein, “pathological condition” or shall refer to an excitotoxic neural injury, epilepsy, ischemia, Alzheimer's disease, traumatic brain injury or any other pathological condition wherein abnormal zinc levels are symptomatic of the pathology.

The present invention provides an implantable fiber-optic zintrode or zinc-sensing probe or zintrode providing real-time monitoring of intracranial Zn⁺² and methods of use. The zinc probe is useful for, but not limited to, basic research on zinc-containing neurons for emergency room and intensive care unit monitoring of acutely brain-injured patients. Additionally, the zinc-sensing zintrode is useful for monitoring brain zinc levels during chelation therapy of traumatic brain injury and of neurodegenerative diseases or other pathological conditions characterized by abnormal levels of zinc.

Measurement of free zinc levels is a technical challenge due to their low physiological level and the presence of interfering cations such as magnesium and calcium. However, an apocarbonic anhydrase-based zinc biosensor provides the sensitivity and specificity required to make such determinations. For example, whereas K_{D} of zinc for wildtype human CA II protein is 4 pM, it is 10 Mm for calcium and 50 mM for magnesium (43). The zinc biosensor has demonstrated a sensitivity of about 0.8 mmoles Zn⁺² in 100 μl volume, equivalent to 800 picomoles or 5×10¹⁴ zinc ions. This sensitivity represents a 100-fold improvement over previous methods for sensing zinc in biological media.

The carbonic anhydrase-based biosensor/zintrode described herein was used to determine the very low in vivo physiological level of extracellular zinc in a resting brain and the level of zinc released from brain tissue in pathological conditions, e.g., ischemia, trauma hypoperfusion and nitric oxide stimulation. The physiological levels of free Zn⁺² in the normal brain ranged from 0.5 nM to 5.0 nM in rabbits with a 1 nM average level. The average 1 nM “baseline” level for free zinc in the brain establishes the true, resting level of extracellular zinc in the rabbit brain and establishes the lower limit of detection required of a zinc biosensor. Clinically, this is the physiological level to which devices or drugs should buffer the brain zinc. During ischemia, free zinc is released as an average surge of about 200 nM.

The zinc probe may be inserted through a ventriculostomy beside the traditional intracranial pressure probe. In acutely brain-injured patients, rising levels of Zn⁺² would alert surgical, ER or ICU staff to intervene aggressively with zinc-release blockers, zinc chelators, zinc channel blockers or other therapies against zinc-mediated neurotoxicity. The zinc probe utilizes an enzyme-based biosensor, e.g., carbonic anhydrase, in the presence of or conjugated to a fluorescent reporter dye, which is incorporated into a zinc-sensing cassette for detection of zinc in complex biological media. The cassette further may comprise the optical components required for fluorescence detection from the fluorophore.

For a carbonic anhydrase-based zinc biosensor or probe, the fluorescent reporters comprising the zinc biosensor must bind to the CA protein; must shift emission max wavelengths in response to the presence of free zinc and must demonstrate an increase of fluorescent intensity in response to the presence of free zinc. Suitable fluorescent reporters may be, although not limited to, dansylamide (DNSA), dapoxylsulfonamide (DPSA) and butyl-DPSA. Furthermore, a fluorescent reporter-apoCA conjugate must not demonstrate non-specific fluorescence in brain dialysate. Thus, ABDN, a widely used fluorophore in zinc sensing (44), cannot be used as microdialysates of brain tested for non-specific fluorescence of the ABDN-apoCA conjugate showed that small molecules in the brain dialysate yielded a fluorescent conjugate with ABDN, apoCA and oxygen with fluorescence in the region of that of the ABDN-apoCA-zinc conjugate (data not shown).

Of the potentially suitable fluorescent reporters, dapoxylsulfonamide has the greatest utility, as it has both (i) tighter binding to holoCA, i.e., low picomole versus low micromole, and (ii) a larger increase of fluorescent yield upon binding to holo-CA, for example, a 20-fold increase versus a 6-fold increase for dansylamide. A large increase in fluorescent yield is critical because of the very low, i.e., 1 nM, physiological level of zinc in the brain. DPSA has an absorption λmax at 340 nm, necessitating UV-transmitting fibers and optics. Characteristics of potential fluorescent reporters are listed in Table 1.

TABLE 1
Reporters for incorporation into CA-based zinc biosensor

| | Abs max | Emm max | Molar ε | |
|---|---|---|---|---|
| Reporter | (nm) | (nm) | (cm⁻¹M⁻¹) | Comments |
| ABDN | 430 | 600 | 8.8 × 10³ | Non-specific |
| | | | | fluorescence problem |
| | | | | in brain dialysates |
| DNSA | 332 | 560 | 4.7 × 10³ | Excellent at low nM |
| | | | | zinc, UV only |
| DPSA | 355 | 605 | 2.2 × 10⁴ | Excellent at low pM |
| | | | | zinc, Biggest increase |
| | | | | in emission with |
| | | | | holoCA; UV excite; |
| | | | | congeners may have |
| | | | | visible excitation |
| Butyl-DPSA | 372 | 560 | 2.7 × 10⁴ | DPSA congener |

A unique attribute of sensors incorporating biological macromolecules as transducers is their capability for modification by site-directed mutagenesis (45-46). Mutant carbonic anhydrase proteins, using the DPSA reporter, for speed of “on-rate” for zinc, to allow monitoring of zinc kinetics and for affinity for zinc, with high affinity desired for measurement of low levels of zinc and moderate affinity desired for measurement of high levels of zinc. CA mutants can be engineered for varying selectivity to zinc relative to other metal ions, e.g. copper. Although not limited to such, carbonic anhydrase mutants may be a E117A mutant, a S166C mutant, or a H36C mutant.

The device further comprises methods of entrapping free zinc utilizing either DPSA or a protein-tethered DPSA or a bead-bound DPSA. ApoCA or mutant apoCA and DPSA may be placed in a microdialysis chamber or “sock,” comprising a semi-permeable membrane that is placed over the tip of an optical fiber and is attached to the fiber, e.g., cementing to the fiber. The membrane is impermeable to apoCA or mutant apoCA and to the fluorophore which will not “leak out” into the biological medium. Free zinc will move freely through the membrane, thus entering the “sock” and binding to the apoCA, which traps it within the “sock”.

Alternatively, the fluorophore may be conjugated to an apoCA. The apoCA-reporter conjugate is then embedded in a water-soluble polymer matrix, such as a solgel, hydrogel or microgel, that is polymerized onto the end of the optical fiber. The solgel pores trap the apoCA-reporter conjugate, while allowing free zinc ions to pass freely. The zinc ions become trapped upon binding to the apoCA. Methods of forming enzyme-reporter conjugates and the sol-gel comprising the conjugate are standard in the art.

The fluorophore may be tethered to a protein, e.g., lysozyme, via a linker. The linker may be pegylated to provide good solubility of the reporter-tether protein conjugate. The tether protein must not reduce the binding of the fluorophore to holoCA and must not quench the reporter fluorescence nor reduce the emission wavelength shift of the reporter upon its binding to holoCA. The reporter-tether protein anchors the reporter within a semipermeable membrane or a solgel matrix.

The fluorophore may be tethered to controlled-pore-glass beads via a carbon linker, e.g., 12- or 14-carbons. The CPG-reporter is placed within the microdialysis membrane “sock” and will bind with a holoCA formed by inward dialyzing zinc ions. The fluorescence of a CPG-linked reporter is not effected by glass beads.

It is contemplated that highly non-polar fluorophores, e.g., DPSA, are physically bound to a small polyethylene thread in an aqueous solution within the cassette. The highly non-polar reporter would physically bind to the polyethylene surface. When a measurement is to be made, the thread is drawn from the cassette and is inserted into the microdialysis “sock”. Again the fluorophore would conjugate to the holoCA formed by inward dialyzing zinc ions.

It is contemplated further that the reporter is chemically bound to a mutant CA protein via a cysteine-thiol attachment point inserted into the CA by directed site point mutagenesis. The cysteine mutation is combined with whatever mutation is required for optimal protein binding and conjugate fluorescence. The reporter-detector linkage, such as a DPSA-CA linkage, may be optimized prior to immobilization of the reporter-apoCA. The reporter-apoCA may be immobilized, inter alia, at the end of a fiber zintrode or at the bottom of a well on a well-plate.

The present invention also provides zinc-sensing zintrodes utilizing mechanical entrapment of free zinc ions on or proximate to a fiber optic. The zintrodes may comprise a microdialysis membrane or “sock” which is placed over the tip of the fiber optic and held in place via heat shrinkable teflon tubing. Alternatively, microdialysis tubing is slipped over an injection-molded optically transparent polymer to create the microdialysis “sock”. Heat shrinkable teflon tubing is used to clamp or seal the microdialysis “sock” around the polymer form. In either zintrode, the reagents, i.e., the apoCA and the reporter, are placed within the “sock”, either separately or in a conjugated form described herein, prior to heat shrinking the teflon.

A zinc-sensing zintrode also may comprise a fiber optic having a U or hairpin shape with a V-shaped notch at the U of the fiber. The reagents are sealed within the V-notch by a semipermeable microdialysis membrane glued onto the fiber. The apoCA and the reporter may be those molecules described herein.

A 370 nm LED illumination source is utilized to emit the excitation wavelength and two photodiode detectors are utilized to detect the emission maxima wavelengths, i.e., 470 nm and 560 nm for dansylamide. This provides ratiometric determination of zinc concentration in a sample. Optionally, depending on the reporter used, bandpass filters selected to pass the emission of the reporter, may also be used. The optical components may be incorporated into a base unit into which the zintrode is positioned. For those zintrodes which are placed proximate to the fiber optic, the fiber optic also comprises the base unit.

Additionally, it is contemplated that the zintrode may use a non-enzymatic zinc-sensing system. These zinc-sensing zintrodes or zintrodes may use a zinc-specific fluorescent reporter dye, e.g. a zinpyr dye (U.S. 2002/0106697), a TSQ or a rhodafluor dye (U.S. 2003/0008405). The dye is dissolved or dispersed into a highly viscous liquid or gel such as, but not limited to, a silicone oil or a viscous alcohol. The fluorescent dye, in its dissolved or dispersed form, is mechanically trapped within the tip of a capillary tube, through which the optical fiber is passed so that the optical fiber tip is located at the tip of the capillary containing the dye solution. The capillary probe is immersed in the biological sample fluid whereupon free zinc diffuses to the probe tip and combines with the fluorescent dye at the probe/sample interface. The characteristic fluorescence of the dye-zinc ion complex is thereby generated.

Zinpyr dyes also may be attached to a solid support via covalent bonds or via hydrogen bonds, either being an alternative to solution based Zn(II) sensors. ZPs may be tethered to a solid, such as glass or quartz, through the bottom ring of the fluorescein backbone since this usually has a minimal effect on the photophysical properties of the sensor (47-48). Alternatively, ZPs or a TSQ dye, such as TFLZn, a strongly ionizing congener of TSQ, may be incorporated into a water soluble polymer matrix, e.g., a solgel or a hydrogel, such as a microgel, via an extended hydrogen bonding network. These types of polymer coatings exhibit a strong adherence to glass or quartz surfaces and are adaptable as support matrices for sensors and optical probes.

To accurately monitor concentration changes, a sensor should have a K_{D} value near the median concentration of its target analyte, i.e., the linear portion of signal versus the analyte concentration plot. For the detection of low levels of physiological zinc, e.g. basal zinc levels in brain, ca. 0.1-1 nM, the fluorescent reporter in the zintrode may be a zinpyr dye such as 9-(o-carboxyphenyl)-2,7-dichloro-4,5-bis[bis(2-pyridylmethyl)-amino methyl]-6-hydroxy-3-xanthanone (ZP1), 9-(o-carboxyphenyl)-4,5-bis[bis(2-pyridylmethyl)-aminomethyl]-6-hydroxy-3-xanthanone (ZP2), (ZP3), 9-(o-carboxyphenyl)-2-chloro-5-[2-{bis(2-pyridylmethyl) aminomethyl}-N-methylaniline]-6-hydroxy-3-xanthanone (ZP4), or (ZPN) or may be TSQ or a congener such as TFLZN. For the detection of moderate levels of physiological zinc in, for example, abnormal physiological states such as ischemia, the fluorescent reporter in the zintrode may be a rhodafluor dye, e.g., (1-[9′-(o-carboxyphenyl)-6′-amino-2′-chloro-3′-xanthanone]-4,10-(diethyl)-7-(2-pyridylmethyl)-1,4,7,10-tetraazacyclododecane (RF2).

The Zinpyr fluorescent sensors utilize fluorescein as the reporting group and are highly sensitive and specific to Zn⁺² (49). The Zinpyr sensors have their excitation and emission wavelengths in the visible range, allowing use of inexpensive excitation and collection optics and avoiding UV degradation of cells in intracellular monitoring applications. ZP1, ZP2, ZP3, ZP4 and ZPN have high affinities for zinc, with K_{D} values between 0.5-1 nM. Ca⁺² and Mg⁺² ions, ubiquitious in biological systems, do not produce measurable changes in fluorescence intensity of the Zinpyrs. Cell permeability of the Zinpyr molecule is high thus facilitating use in intracellular measurements of zinc. Zinpyr quantum yield is high, i.e., about 0.25-0.4, and binding of zinc to the Zinpyr dyes causes a three- to six-fold enhancement of fluorescent intensity under simulated physiological conditions, along with a slight hypsochromic shift in excitation wavelength maximum, e.g. from 515 nm to 507 nm for ZP1.

For example, ZP4, a second generation Zinpyr sensor, has a lower pKa value than the first generation Zinpyr 1 and Zinpyr 2 sensors, making it less sensitive to protonation of the tertiary amines which are responsible for the increased fluorescence of Zn⁺² binding, under physiological conditions, and diminishing background fluorescence of the unbound Zinpyr molecule. Zinc binding increases the quantum yield of ZP4 from about 0.06 to about 0.34 compared to a change in ZP1 from about 0.39 to about 0.87. The reduced background fluorescence of the unbound ZP4 improves measurement of Zn⁺² concentration gradients, as compared to ZP1 and ZP2.

The rhodafluor fluorescent sensors utilize a hybrid of rhodamine and fluorescein as the reporting group. Like the Zinpyr sensors, the rhodafluors have their excitation and emission wavelengths in the visible range, allowing use of inexpensive excitation and collection optics. The increase in fluorescence intensity upon zinc binding is less for rhodafluors than for the zinpyr reporters. However, unlike the tight-binding, i.e., sub-nM K_{D}, zinpyr sensors, the rhodafluors have moderate affinities for zinc, with K_{D} values in the range of about 10-20 μM. This allows their use in applications in which tight-binding sensors are inadequate, such as measurement of μM zinc levels observed in abnormal physiological states. The rhodafluor sensors are ratiometric, an advantage over the zinpyr sensors.

Kits comprising the zintrode also are provided. The kit may comprise at least one zintrode in a self-contained cassette or cuvette. The cassette or cuvette may contain a zinc calibration solution. The kit may further comprise the optical components required to determine zinc concentration in a sample. For example the kit may comprise a base unit containing the necessary optical components into which the zintrode/cassette may be positioned.

Also provided is a multitrode device utilizing an existing Optex tritrode and the U-shaped zinc-sensing zintrode provided herein. The tritode comprises three separate zintrodes or channels to monitor pH, oxygen and carbon dioxide in a patient. The multitrode further incorporates arterial access housing for the zintrodes, e.g., an arterial introducer or a cannula. The multitrode also incorporates a base unit comprising the hardware to monitor the zintrodes.

The tight U-shaped bend in each fiber optic provides true transmission measurements of the emission signal. This is an improvement over previous zintrodes which utilized a mono-fiber arrangement in which light travels in both directions, i.e., excitation light to the sensor and emission light from the sensor. Addition of the zinc-sensing zintrode provides a multitrode device which provides a simple and accurate means to monitor these four critical parameters in times of accidents or other emergencies thus accommodating the anticipated needs of critical care patients in hospitals.

The present invention also provides methods of monitoring and buffering zinc levels within the lower and upper thresholds of the basal range in emergency room and ICU situations using the zintrode or multitrode described herein. Such methods may be used to treat pathological conditions such as excitotoxic neural injury where the zinc level in the brain is monitored in real-time. During monitoring, treatment parameters are adjusted to maintain the level of free zinc in brain between 0.1 nM and 20 nM, more preferably between the lower and upper threshold levels of 0.5 nM and 5 nM and most preferably to about 1 nM, in order to avoid epileptic seizures at the low end and neurotoxicity at the high end.

Excessive levels of free zinc may be controlled with zinc-release blockers, zinc chelators, zinc channel blockers or other therapies against zinc-mediated neurotoxicity. It is contemplated that free zinc levels are buffered within the 0.5 nM and 5 nM range, most preferably to about 1 nM, by administering a pharmacologically effective amount of a zinc chelator such as, but not limited to, clioquinol. Clioquinol, is a membrane permeable zinc chelator which, in a therapeutically effective dose determined from the readings obtained from the zintrode or multitrode, chelates an amount of free zinc sufficient to maintain an appropriate level within the brain. It is well within the scope of one of ordinary skill in the art to determine dose of such therapeutic agents.

The present invention also provides a pZn meter. The pZn meter may measure pZn in synthetic salt solutions, such as Ringers, ACSF, and other buffered salt solutions used for brain cells or tissues. Additionally, the meter may measure pZn in biological solutions, such as, but not limited to, a growth media with fetal calf serum, CSF, blood or seminal serum, saliva, tears, urine, or synthetic salt solutions in which tissue has been placed, such as perfusate from a brain tissue chamber. For measuring pZn in biological solutions or fluids, it is necessary to remove essentially all organics, cleaning up the sample by dialysis through our MW100 cutoff dialysis membrane, to remove essentially all organics. It is contemplated that a clean-up cuvette comprising the MW100 cutoff dialysis membrane is provided with the pZn meter or individually in a kit.

As described herein, the invention provides a number of therapeutic advantages and uses. The embodiments and variations described in detail herein are to be interpreted by the appended claims and equivalents thereof. The following examples are given for the purpose of illustrating various embodiments of the invention and are not meant to limit the present invention in any fashion.

### EXAMPLE 1

Measuring pZn and Determining the Baseline or “Resting” pZn in the Brain with apoCA

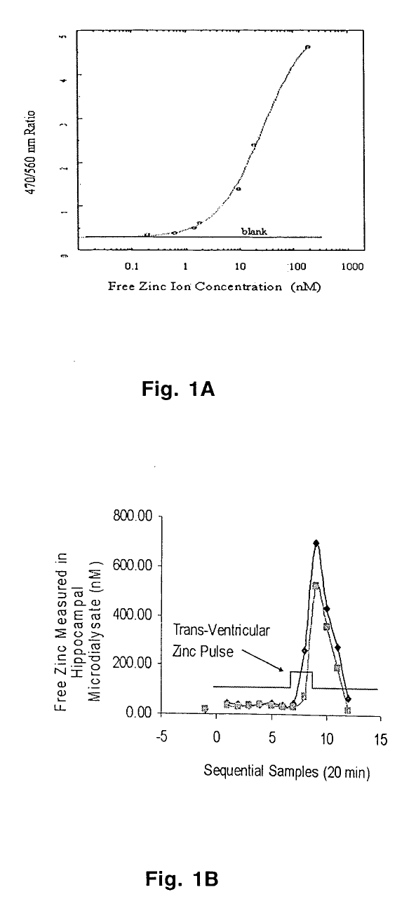

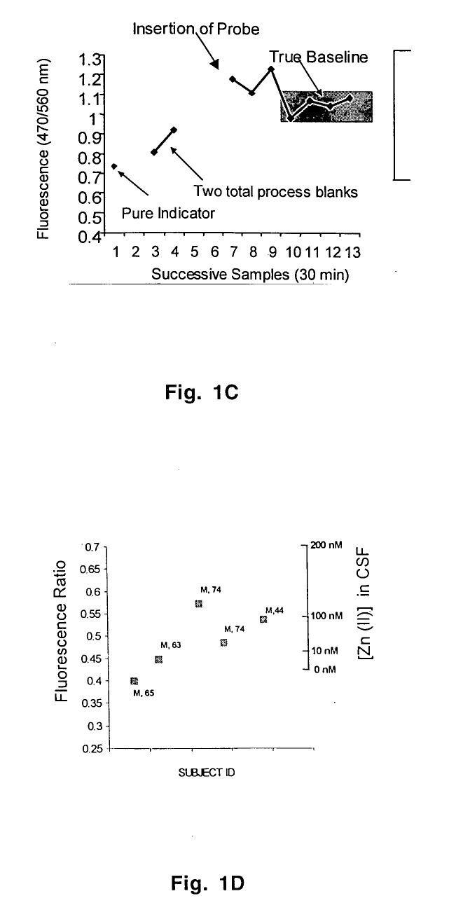

Microdialysis probes were implanted into the brains of acutely prepared rats and rabbits. A ligand trapping strategy was used to avoid not measuring all available free zinc. The dialysis fluid was loaded with apoCA and that fluid then was used for the perfusions. The 30,000 D protein did not leave the dialysis tube due to the MW cutoff 10 KD membrane. The dialysate was collected and the concentration of holoCA, and thus the concentration of the formerly free zinc in the dialysate, was measured by simply adding in an appropriate fluorescent reporter and performing a ratiometric measurement of the holoCA concentration in a cuvette with a SPEX Fluorlog III. Phantom brains, i.e., beakers, were used as a calibration standard. The beakers were filled with known concentrations of zinc and dialyzed as were the brains. Calibration curves corrected for recovery were generated (FIG. 1A).

Free zinc in the rat brain also was determined. Probes were aimed at the dorsal hippocampus while the ventricular system of the rat was perfused with a wash of either plain artificial CFS (ACSF) or ACSF with 200 μM zinc. Perfusion fluid went into the 3^{rd} ventricle at 2 μL/min and came out through a tube penetrating the nuchal ligament and dura. Perfusing the ventricle with a high zinc solution for 40 min caused an abrupt rise in the free zinc concentration in the dialysate. The shape of the zinc rise measured by dialysis reflects both the diffusion time through the brain and the brain's capacity to partially bind, i.e., buffer, the exogenous free zinc. The abrupt rise in zinc in the dialysate during the zinc “pulse” is shown for two rats in FIG. 1B.

A rat was anesthetized with isofluorane and a dialysis probe (200 μm diam; 2 mm exposed membrane) was implanted and aimed at the dorsal hippocampus. Sampling was done at 1 or 2 μL/min with 20 to 100 μL samples being collected serially for three hours. An insertion spike in the zinc level appeared upon inserting the probe into the brain, however, the baseline was relatively stable for the last two hours of sampling (FIG. 1C). This was repeated in rabbits, using the same methods as for rats, except that blood gasses, EEG and respiration were monitored to allow maintenance of a stable physiological condition (data not shown).

Free zinc also was determined in cerebral spinal fluid (CSF). Five samples of human CSF were collected from spinal taps and assayed using the apoCA-microdialysis method, as used for the rats and rabbits described above. The lowest two values, one of which could not be resolved from the “0” blank, are below 10 nM, also as demonstrated in rats and rabbits. It is assumed that the higher values reflect hemolysis or other contamination introduced durng sample collection. This appears similar to the “insertion spike” seen in experimental animals (FIG. 1D).

In approximately 80% of all determinations, a brief, i.e., 20-60 min, “spike” in the level of free zinc in the dialysates appeared immediately after probe insertion. Though this was absent in a few, i.e., ˜20%, rats or rabbits, it generally was present and is attributed to the initial outflow of zinc from damaged glia, endothelial cells, neurons or other damaged cells. A comparable “flood” of other analytes, e.g., glutamate, typically is seen in acute microdialysis experiments at the time of probe insertion (data not shown). Furthermore, after the initial insertion spike of free zinc, the baseline free zinc or pZn typically fell between 0.5 nM and 5.0 nM (pZn=8.3 to 9.3). This range has been demonstrated repeatedly. It is contemplated that the between-animal variability is real biological variability probably related to the exact placement of the probe and the nature and extent of the brain injury caused upon insertion of the probes.

### EXAMPLE 2

Release of Zinc During Excitotoxic Brain Insult

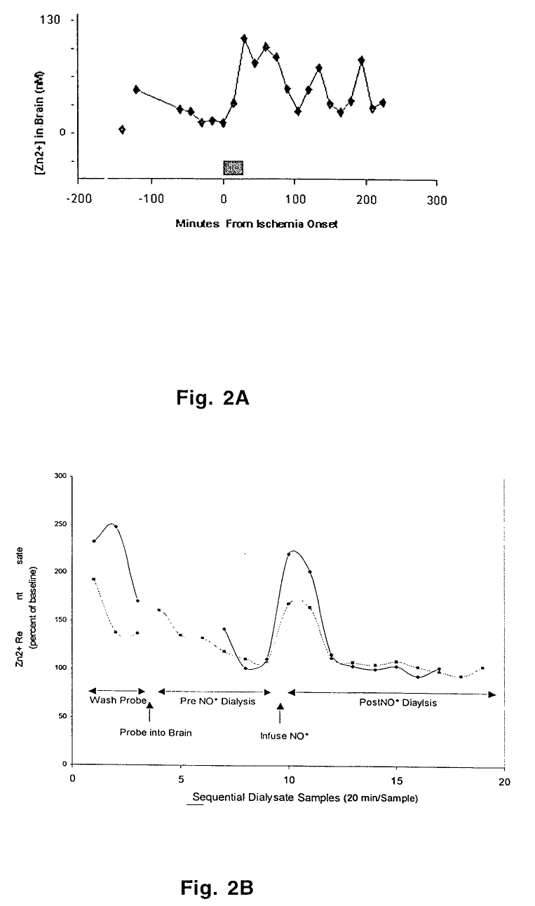

Ischemia caused a release of free zinc in the brains of rabbits and rats with the high level of zinc remaining throughout the reperfusion that follows as treatment for ischemia. Ischemia and reperfusion injury in more than a dozen rabbits was induced using a whole-neck cuff method (Bacher, 1997 3420/id). The apoCA microdialysis method was used to monitor free zinc before and after a 20 min ischemia in the anesthetized rabbit. The zinc “spikes” upon probe insertion settle to a value close to 1 nM, then rises abruptly at ischemia onset and stays high for 90, fluctuating thereafter from high to low for 4 hours (FIG. 2A).

What is more notable than the rise during ischemia is the later, variable, but long-lasting rise observed upon reperfusion. This slow variable, lasting release of zinc after the start of reperfusion varies considerably in timing among individual rabbits and it is not known what determines the variability among the tested rabbits. This may reflect the various sequellae of the ischemia, for example, but not limited to, seizure activity, BBB and blood flow changes, and pH changes. However, this reperfusion-induced release virtually is identical to that seen in brain slices in vitro after reperfusion.

Additionally, it has been demonstrated abundantly that the excitotoxic release of zinc, as seen in ischemia, for example, is partly driven NO*. A similar zinc release, though higher in magnitude is caused by merely infusing NO* into the rabbit brain. The microdialysis probes were aimed at the dorsal hippocampus in two acutely prepared rabbits, as described above, and the rabbits were anesthetized with isofluorane. Throughout the procedure the rabbits were monitored to maintain stable heart rate, PO₂, PCO₂, and normal amplitude cortical EEG.

After allowing the rabbits to lie undisturbed for 2 hours after probe insertion, a microvalve was switched that caused the microdialysate fluid to change from the standard ACSF+apoCA to the same fluid with 1 mM spermine NONOate mixed in seconds before switching the valve. This caused spermine NONOate to release NO*, some of which presumably passed through the dialysis membrane and into the brain as a reverse dialysis procedure. The infusion lasted 10 minutes.

Both rabbits demonstrated essentially the same vigorous release of free zinc that lasted through 2 samples, i.e., throughout the infusion and for part of the first 20 min in following the infusion (FIG. 2B). The measured free zinc in the spermine NONOate solution was slightly higher than in the drug free solution, but that degree of contamination was only about 20% as high as the value of free zinc observed in the dialysate. Although unlikely, it is possible this is caused by spermine releasing zinc.

### EXAMPLE 3

Measuring Zinc with apoCA-Based Fiber Optic Zintrode

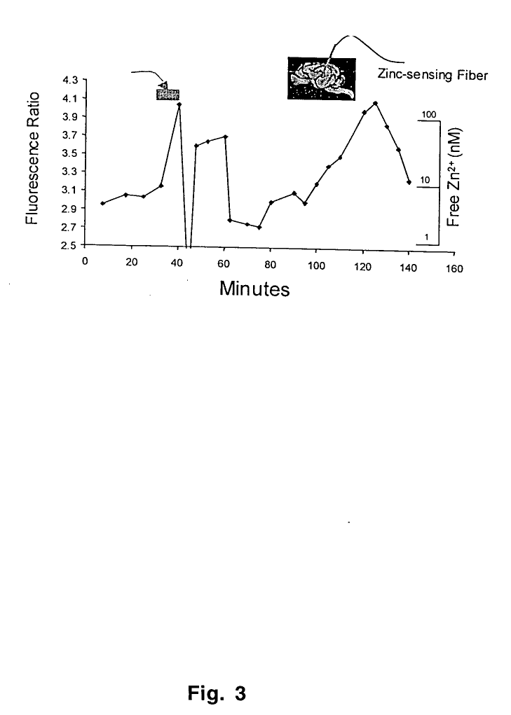

An apoCA-tipped optical fiber was inserted into the neocortex of an anesthetized dog. After collecting baseline, the dog was subjected to cardiac arrest followed by restarting the heart. As with rabbits, the free zinc rose immediately and then showed a delayed, long-lasting post-reperfusion rise in free zinc (FIG. 3). A brief drop in the free zinc reading at about minute 45 is unexplained, but the apoCA probe is demonstrably sensitive to pH, with low pH reducing the apparent [Zn2+], due to reduced binding of the apoCA at low pH. To get corrected free zinc values, an optical fiber pH sensor may be used beside the apoCA sensor to allow the pH effect to be corrected for.

### EXAMPLE 4

Screening Fluorescent Reporter Candidates

Stock solutions of the fluorescent reporter were prepared in alcohol and diluted 1000-fold in the final measuring cuvette. Carbonic anhydrase protein (CA) was “apo-ized” as per a standard procedure (50). Zinc was added to a solution of the CA in the presence of the reporter and the change in fluorescence intensity and wavelength of the reporter were measured on a Spex DM-1B fluorescence spectrophotometer using He—Cd laser excitation.

### EXAMPLE 5

Mutant Carbonic Anhydrase Proteins

The three mutants yielded an approximate 2-fold increase in emission intensity relative to DPSA alone and also had acceptably large wavelength shifts. Whereas 1 μM DPSA without any CA yielded 70 fluorescence intensity units as a baseline intensity, 1 μM DPSA with wildtype CA-Zn yielded 300 units. 1 μM DPSA with three mutants yielded 250 units with S166C mutant, 200 units with H36C mutant and 170 units E117A mutant. The E117A CA mutant had an 80-fold faster “on-rate” for zinc than did wildtype CA, allowing more accurate measurement of zinc release kinetics. In the ZincDetect system, this will allow use of 80-fold less of the CA protein in the microdialysis cassette, with no loss in the total amount of zinc ion which is “captured” by the CA protein. The E117A CA mutant is also useful for probe applications requiring detection of rising zinc levels as fast as possible, as required in ER trauma situations.

Another CA mutant, E117A, had a reduced affinity for zinc, i.e., micromolar K_{D} versus picomolar K_{D} of wildtype CA, allowing for accurate measurement of high zinc levels. The preferred reporter, DPSA, was also tested for affinity for both the wildtype CA and the CA mutants to ensure that the engineered CA mutants did not exhibit a reduction in affinity for DPSA, relative to that of wildtype CA. The screening experiments demonstrated that both the wildtype CA and the mutant CA proteins can be used in combination with the DPSA reporter to measure zinc levels in various biological systems.

### EXAMPLE 6

Measurement of K_{D} of Fluorophore for CA Mutant

The measurement is based on a literature method (44, 51). Aliquots of a 1 μM solution of the CA protein are mixed with several concentrations of DPSA, ranging from 1 nM to 10 μM. After 30 minutes of incubation, the fluorescence emission spectrum is measured with excitation at 365 nm and emission at 400-700 nm. Taking the intensity at 560 nm, which is the emission max of an apoCA-DPSA conjugate, at the highest concentration aliquot to represent 99% fractional saturation, and 1% at the lowest concentration aliquot, the fractional saturation is plotted as a function of the DPSA concentration on a logarithmic scale, and the data is fit to a single binding isotherm, e.g. fraction bound=K_{D}/(1+K_{D}) using Kaleidagraph (Synergy Software), or a similar program.

Because of the nature of the sulfonamide binding as a fourth ligand to the active site, DPSA binds as a 1:1 complex at realistic aliquot concentrations. If the fractional saturation is not near 1.0 at higher DPSA concentration because the DPSA binds more weakly to the variant, the curvature of the isotherm will not reach the peak intensity. If binding is much tighter, then this is demonstrated by the lack of free DPSA, which is apparent from the spectrum, at lower DPSA concentrations and the non-saturating increase in intensity with DPSA concentration up to saturation of the protein.

### EXAMPLE 7

Preparation of Carbonic Anhydrase Mutant

Oligonucleotide-directed mutagenesis of the cloned human CA II gene in plasmid Pcam-al was performed as previously described (45). Plasmids encoding CA II variants were transformed into E. coli strain BL21(DE3) cells following a method as described previously (52). Colonies with plasmids encoding the mutant gene were identified using esterase and sulfonamide binding activity screens (45).

Protein expression and purification was achieved by growing E. coli cells containing the Pcam-al encoding the CA II gene and inducing CA II synthesis by addition of 1 mM isopropyl-β-D-thiogalactopyranoside and 0.3 mM ZnSO4, followed by incubation at 31-34° C. for 5-6 hours. Cells are pelleted and lysed with EDTA/lysozyme in the presence of protease inhibitors, followed by removal of cellular remnants by centrifugation at 16,000 g for 45 minutes. The protein was then purified by affinity chromatography at pH 8.0. Protein samples were then extensively dialyzed against 5 mM potassium phosphate buffer, lyophilized, and stored at −20° C.

### EXAMPLE 8

Preparation of Enzyme

The apo form of the CA II wildtype and mutant proteins was prepared as previously described (45). Special care was taken to limit exposure of the CA protein to metal ions. All buffers and solutions were prepared using deionized water and stored in plasticware previously treated with 5 mM EDTA solution to remove trace metal ions. Metal-free apo-CA II was prepared using Amicon diaflow filtration, washing first against 50 mM dipicolinate (DPA, Sigma) at Ph7.0 and then against 10 mM ACES Ph7.0. Excess DPA was removed by gel filtration chromatography on a Sephadex PD-10 column (Pharmacia).

### EXAMPLE 9

Mechanical or Molecular Entrapment of Free Zinc Ions in apoCA Zintrodes

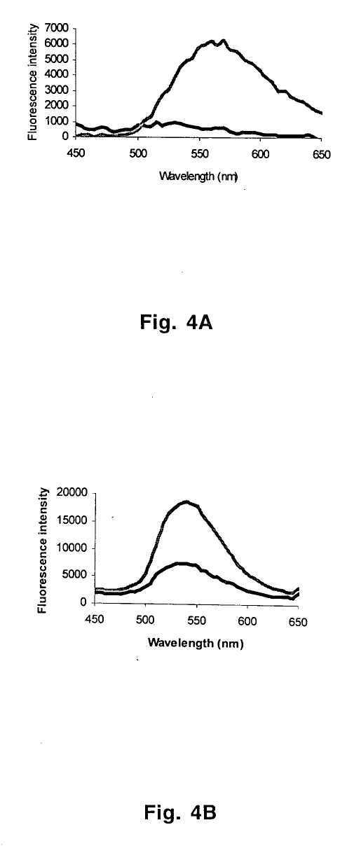

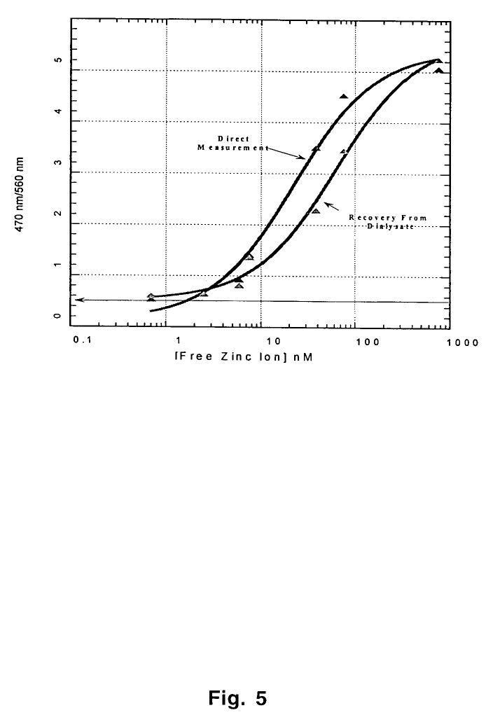

A solution of DPSA was placed in the “sock” and the fluorescence of DPSA inside the dialysis chamber and in the external HEPES buffer solution was measured (FIG. 4A). The chamber was then filled with 10 μM apoCA and the external solution with 50 μM HEPES buffer and 10 μM free zinc (FIG. 4B). Since the microdialysis membrane is impermeable to apoCA, any zinc that passes through the membrane is then trapped inside upon binding to apoCA. Recovery measurements, using wildtype apoCA and a DNSA reporter, indicated that 45% of the free zinc is trapped within the dialysis “sock” (FIG. 5). For example, if free zinc in the biological medium is 2 nM, then 0.90 nM zinc will be trapped within the “sock.”

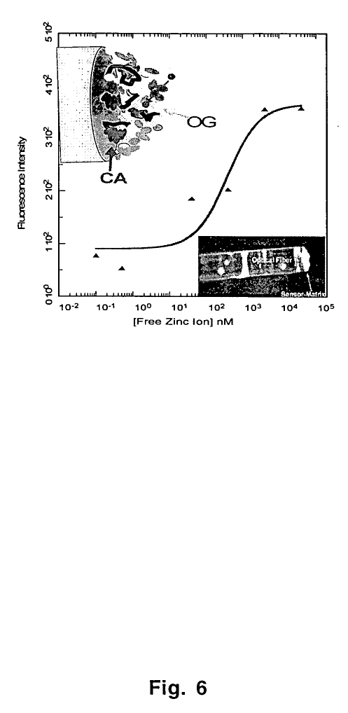

Alternatively, free zinc ions may pass through a solgel matrix polymerized onto the end of the optical fiber. Oregon Green is tethered to apoCA and the apoCA-OG conjugate is embedded within the sol-gel matrix. Zinc ions bind to the apoCA as the ions pass through the sol-gel matrix (FIG. 6). Another example of an apoCA-tipped optical fiber utilizes a polymer of ABDN, polyABDN. The entire polyABDN molecule with the apoCA would be physically trapped when embedded within a solgel matrix. The apoCA, the polyABDN and the solgel are mixed together in solution, then the solution is applied to optical fiber tips by dipping and is allowed to set on the fiber optic tip. These fibers have fast on responses to zinc transients and are sensitive to the clinically-relevant range of free zinc, which is from ˜1 nM for the healthy resting brain up to ˜1 μM for a brain that is undergoing an excitotoxic crisis as demonstrated in the animal models for ischemiz/reperfusion, trauma and seizures.

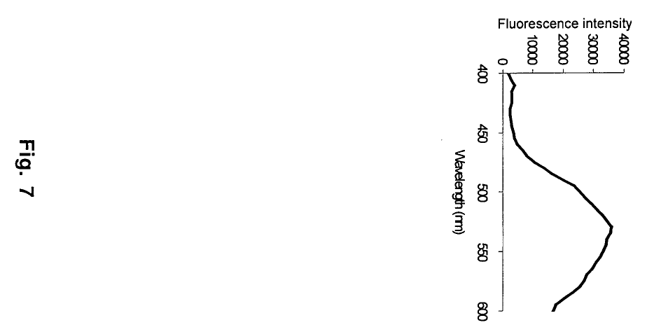

DPSA is membrane impermeable and compatible with the microdialysis membrane or “sock”. However, DNSA “leaks out” of the membrane. DNSA reporter was covalently bound to amino-CPG beads using a 12 C or a 14 C tether and placed into the microdialysis membrane or “sock”. Binding DNSA to CPG beads did not alter its fluorescence. The DNSA-CPG bead system comprises a “molecular tether” means of entrapment (FIG. 7).

### EXAMPLE 10

ApoCA Zintrodes Using Mechanical Entrapment

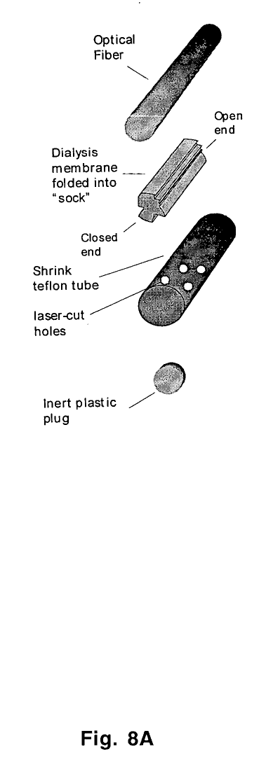

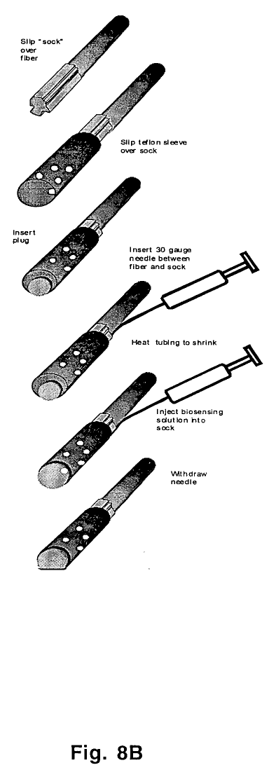

The component parts of one zinc-sensing probe, based on mechanical entrapment of the reagents, are illustrated in FIG. 8A. The assembly of the component parts into the probe is illustrated in FIG. 8B. The microdialysis membrane which is a 100 MW cutoff semipermeable membrane, is folded into a “sock” having a closed end and an open end, and the open end is slipped over the optical fiber. A teflon sleeve, previously laser-drilled with holes, is then slipped over the “sock,” and an inert plastic plug is inserted into the end of the teflon sleeve. A fine hypodermic needle such as a 30 gauge needle is then inserted between the fiber and the “sock” in order to load the reagent solution into the “sock.” The teflon sleeve is then gently warmed, with the membrane still wet, to heat-shrink the assembly tightly together with the microdialysis tube in place.

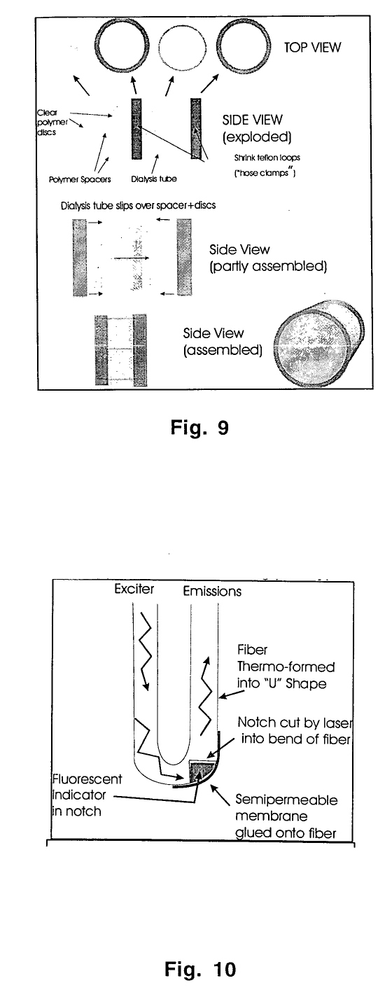

A second zinc-sensing mechanical entrapment probe is depicted in FIG. 9. This zintrode comprises an injection-molded polymer part with a reasonably flat and thin round disk on each end. The disks are separated by 3-5 spacers which position the two disks parallel to each other. A short length of microdialysis tubing is slipped over the two disks and spacers, so as to fit snugly, creating the microdialysis “sock”. Two short sections of shrink teflon tubing are then used as “hose clamps” to lock the microdialysis tubing to the end disks. An adhesive can be used to eliminate leaks, if necessary. The biosensor reagents are syringe-loaded into the microdialysis tubing just before “heat-shrinking” the teflon tubing. The polymer for this assembly is chosen for both optical and chemical properties. The polymer must demonstrate optical transparency and minimal scattering at 300-600 nm and negligible fluorescence in the 400-600 nm region. The polymer must not leach or adsorb metal ions from solution and must not inactivate the CA enzyme.

A third zinc-sensing mechanical entrapment probe or zintrode utilizes a 10-100 micron optical fiber that has been heated into a hairpin or “U” shape (FIG. 10). A laser is used to cut a V-shaped notch into the “U” of the fiber and the notch is then loaded with the reagent solution and covered by gluing a semipermeable microdialysis membrane onto the fiber. When the fiber is immersed in sample solution, e.g. a calibration solution, serum, CSF, or extracellular fluid, the Zn⁺² ions cross the microdialysis membrane freely and bind to the CA protein to form holoCA.

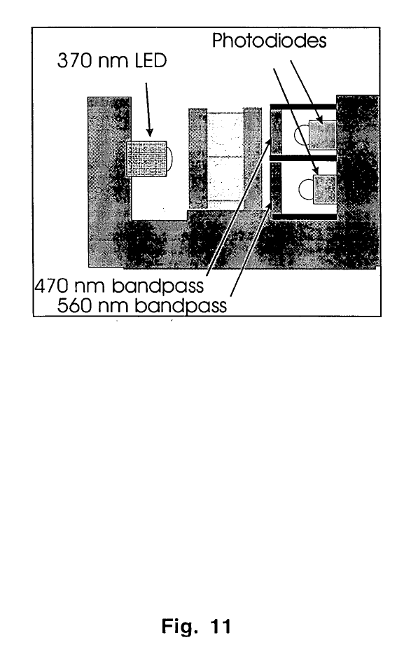

The optical components for the zinc-sensing mechanical entrapment zintrodes require a 370 nm LED illumination source to emit the excitation wavelength and two photodiode detectors and, depending on the reporter used, bandpass filters to the selected wavelengths to pass the emission of the reporter. The optical components are incorporated into a base unit into which, for a DNSA reporter, the optical system would requires 470 nm and 560 nm bandpass filters (FIG. 11). The two photodiode detectors will provide a ratiometric determination of zinc in the sample fluid external to the microdialysis chamber to that of zinc within the microdialysis chamber.

### EXAMPLE 11

Method of Use and Calibration of apoCA-Zinc Biosensor Cassette

A pre-filled refrigeratored cassette is placed into a container holding the sample fluid in which the Zn⁺² is to be analyzed. The container of sample fluid is held at constant temperature of 34° C. with the fluid stirred gently. During this incubation time, zinc moves freely through the microdialysis membrane and is trapped by the apoCA, which at 20 μM concentration has an effectively fast “on rate.” In principle, virtually all of the free zinc that is in the outer container will be trapped by the apoCA and converted to holoCA.

Thus, if a very high zinc concentration is measured or a long incubation time is used, the apoCA will be saturated and the zinc level of the sample cannot be accurately measured. Therefore, the user must do some “range finding” to determine, within 2-3 orders of magnitude, how much zinc is in the sample. Samples far above the convenient capability of the zinc biosensor cassette would then be diluted. Samples below the detection limit of the cassette are unlikely, in that the system can be “tuned” to low picomolar sensitivity, i.e., parts per quadrillion. The knowledge of the physiological level of zinc in the normal brain and of the excess level of zinc in the ischemic brain, obtained with the device described herein, is helpful in “range finding”.

### EXAMPLE 12

Zinpyr or Rhodafluor Zinc-Sensing Zintrodes Using Mechanical Entrapment

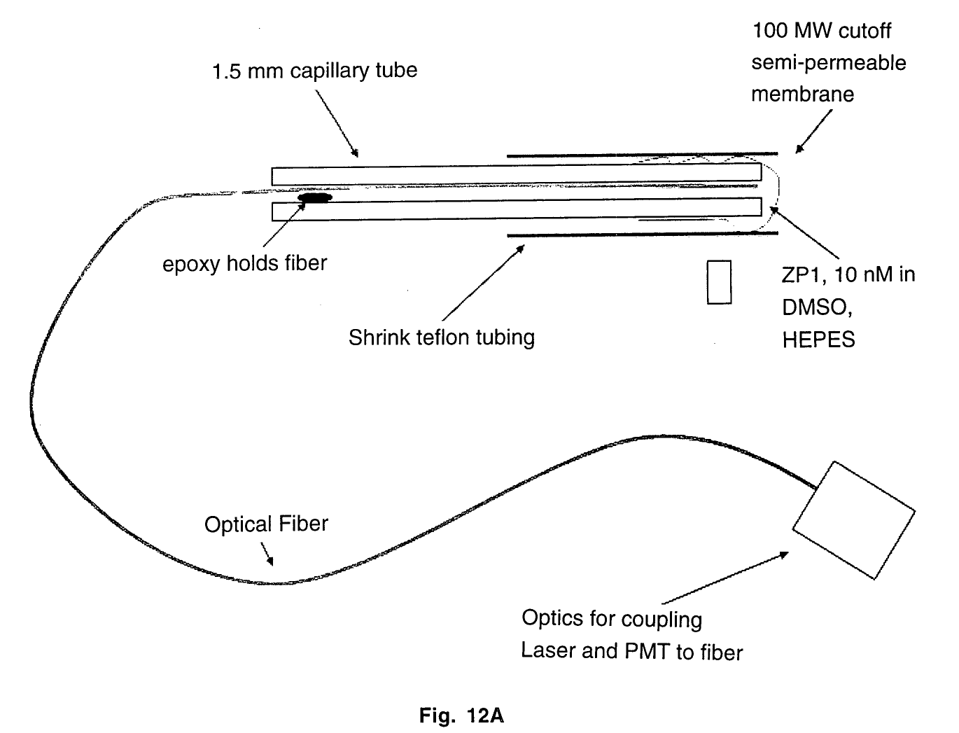

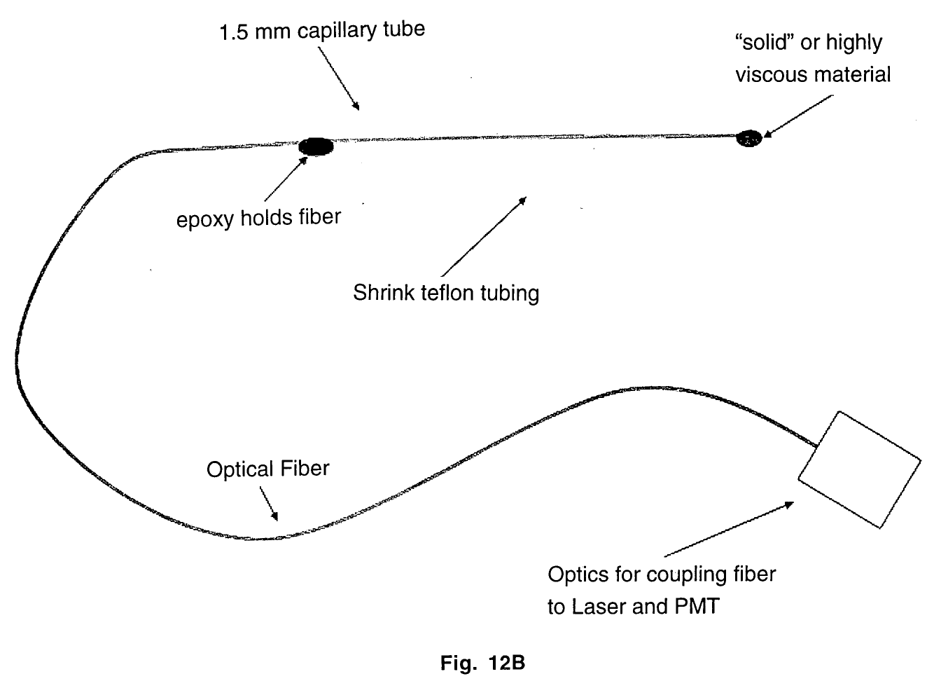

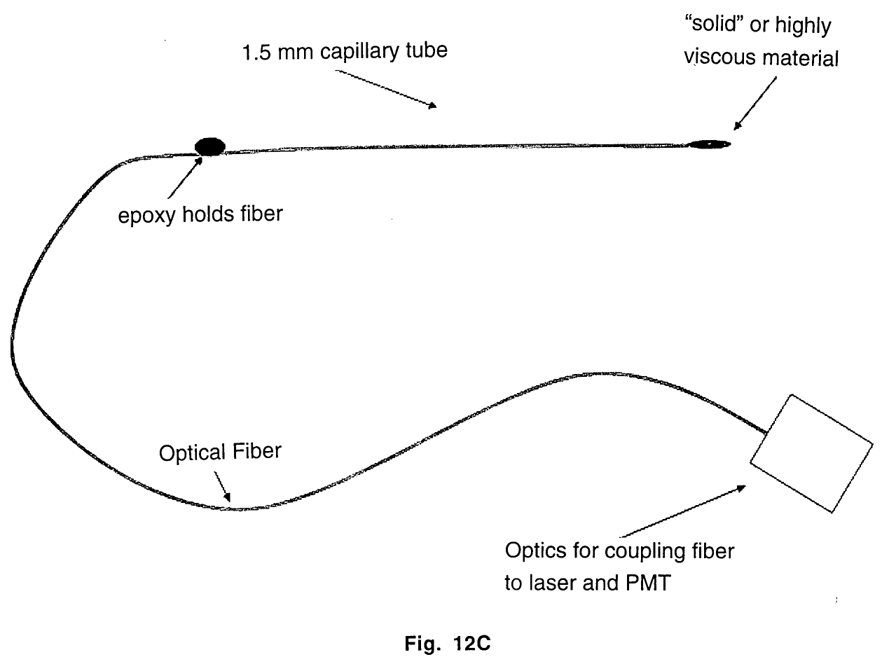

FIG. 12A depicts an zintrode using mechanical entrapment of a fluorescent dye via a semipermeable membrane to form a microdialysis chamber. A fiber optic is inserted down the center of a 1.5 mm capillary tube and the fiber is locked in place at the capillary “entrance” end by epoxy. The capillary is filled with a solution of 10 nM ZP or rhodafluor sensor, such as ZP1 or RF2, dissolved in DMSO and HEPES buffer.

A 100 MW cutoff semi-permeable membrane, that is similar to that used for the mechanical entrapment of apoCA and the fluorescent reporters such as DPSA or DNSA, is placed over the “exit” end of the capillary, encapsulating the ZP solution in a microdialysis chamber. A shrink teflon tube is placed over the semi-permeable membrane so as to secure it to the capillary. The capillary zintrode is immersed in biological sample fluid. Free zinc ions diffuse freely into and across the semi-permeable membrane and enter the microdialysis chamber to contact and complex with the ZP sensor. This causes increased ZP fluorescence which is collected by the optical fiber.

Alternatively, FIG. 12B depicts a modification of the mechanical entrapment depicted in FIG. 12A in which the semi-permeable membrane is not required thereby simplifying probe fabrication. The 1.5 mm capillary tube is filled with a solution of 10 nM ZP or rhodafluor sensor in a highly viscous low polarity liquid, e.g. a high molecular weight alcohol or fatty acid or a silicone oil such as dimethyl silicone oil. The highly viscous sensor solution is held in the capillary tube by surface tension and forms an interface between the end of the capillary and the aqueous biological sample fluid. Free zinc in the sample fluid diffuses to the viscous fluid interface, combines with the dispersed/dissolved ZP sensor turning on its fluorescence, which is collected by the optical fiber. FIG. 12C depicts a further modification of the entrapment depicted in FIG. 12B wherein mechanical entrapment of the viscous sensor solution is improved by “necking down” the “interface” end of the 1.5 mm OD capillary to 50 microns.

### EXAMPLE 13

Covalent Bonding Attachment of Zinpyr Dyes

Zinpyr derivatives may be attached into or onto a solid support through derivitization of the bottom ring of the fluorescein backbone. Furthermore, substitution on the bottom ring of the fluorescein backbone has been extensively explored and a reactive carboxylic acid functionality can be placed on either the 5- or 6-position of fluorescein or of 2′,7′-dichlorfluorescein (47-48,53). This carboxylic acid can be selectively transformed into a reactive acid chloride or ester functionality through precedented chemistry (53). Amines, e.g., 4-aminobutanol, 1,4-diaminocyclohexane and 1,3-diaminopropane, react with these intermediates to yield fluoresceins that are attached to a bifunctional linkage via an amide functionality. Amide functionalities are preferred over esters due to their superior chemical robustness. The linkage precursor will likely be a diamine in order to have amide or amine functionalities at both ends of the tether between the fluorophore and the solid support. Scheme 1 shows the 6-carboxylic acid derivative of 2′,7′-dichlorofluorescein reacting with 1,4-diaminocyclohexane as a sample reaction.

Once the fluorescein linkage complex is made, there are two general synthetic routes that would complete the synthesis of the zintrode. In the first route, the material can react with an appropriately derivatized solid, such as polystyrene copolymerized with maleic acid butyl ester, chlorotrityl polystyrene resin, or chloromethylated styrene, followed by subsequent Mannich attachment of the Zn(II)-binding dipicolylamine moieties (54). Fluoresceins have already been attached to the chloromethylated styrene, although not via the proposed linkage (55-56). Amines are readily linked ot chlorotrityl polystyrene resins (57). In the second route, the pendant amine on the linkage can be protected prior to the Mannich attachment of the dipicolylamines. Subsequently, the amine is deprotected before attachment to the solid support. Since the solid support may be thought of a protecting group, the first general route may be more efficient. These synthetic steps are in Scheme I below.

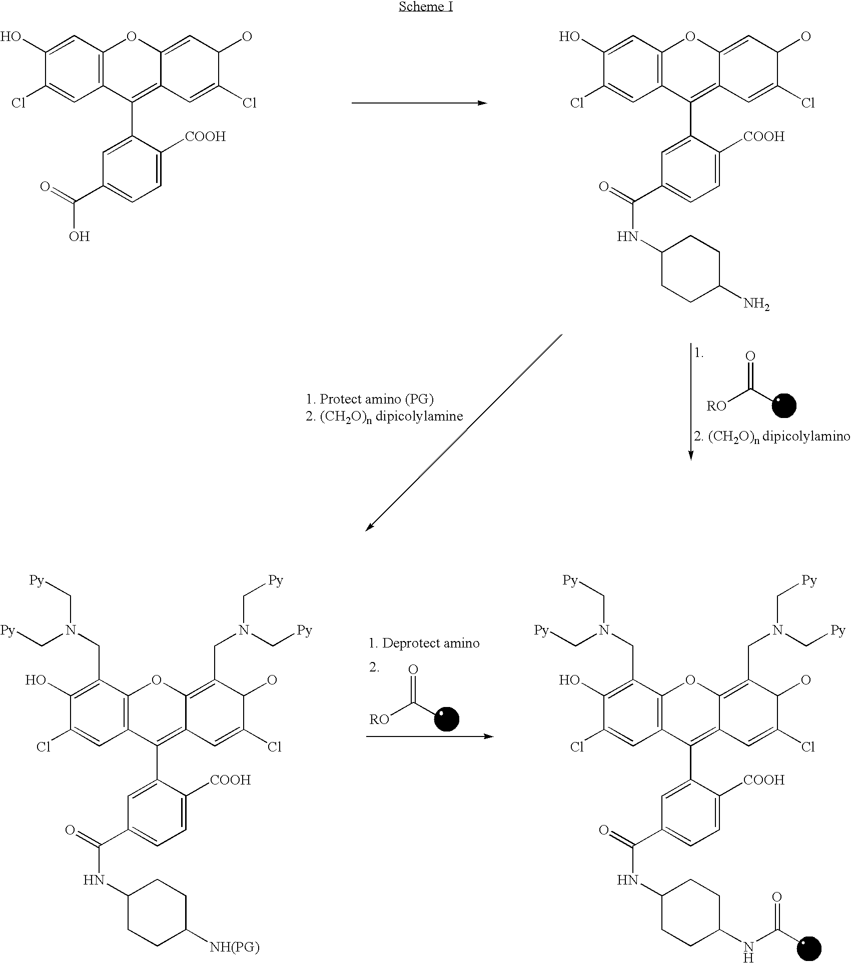

### EXAMPLE 14

Polymeric Attachment (Hydrogen Bonding) of Zinpyr or TSQ Dyes

Monolayer Preparation

All glassware used to prepare the layers is cleaned by sonicating for 15 minutes in a 2%/v Hellmanex II solution in distilled water, rinsed four times with high purity (MilliQ, 18.2 M cm) water, and dried in an oven at 150° C. The substrates, quartz or glass slides and silicon wafers were cleaned for 15 minutes in piranha solution, i.e., concentrated H2SO4 and 33% aqueous H₂O₂ in a 3:1 ratio. They were then rinsed several times with high purity (MilliQ) water and dried in a nitrogen stream immediately prior to performing the formation of the monolayer.

Microgel/Zn-Chelator Formulations

TFLZN (8 mg) was dissolved in aqueous DMSO (200 μL DMSO/200 μL water). To this homogenous solution was added 2 ml of a 2.5% w/w aqueous microgel solution followed by stirring for 2 hours. The resulting homogenous solution was then applied to a clean glass slide in 2.5 μL and 25 μL volumes and the droplets allowed to dry for 24 hours.

ZP1 (5 mg) was initially dissolved in DMSO (1 ml) followed by the addition of 4 ml of a 2.5% w/w aqueous micro solution and the mixture stirred for 2 hours. The mixture was then centrifuged for 30 minutes and the homogenous supernatant separated and applied to a clean glass slide as describe.

ZP4 (10 mg) was added to 2.5 ml of the 2.5% microgel and stirred for 2 hours. The resulting solution was centrifuged and plated as described.

An amine terminated polyurethane microgel, i.e., a low molecular weight hydrogel of about 100 Kda, formulation containing the zinc chelators TFLZn, ZP1 and ZP4 are prepared. The microgel matrix provides terminal amine functionality which incorporates the chelators into the water soluble polymer matrix via an extended network of hydrogen bonding. The formulations are then dip-coated onto an appropriately cleaned glass surface and allowed to air dry for 24 hours. The resulting hydrogel coating retains a high degree of water content and is readily permeable to low molecular weight, aqueous soluble analytes. Formation of the microgel/Zn chelator complex is shown in Scheme II.

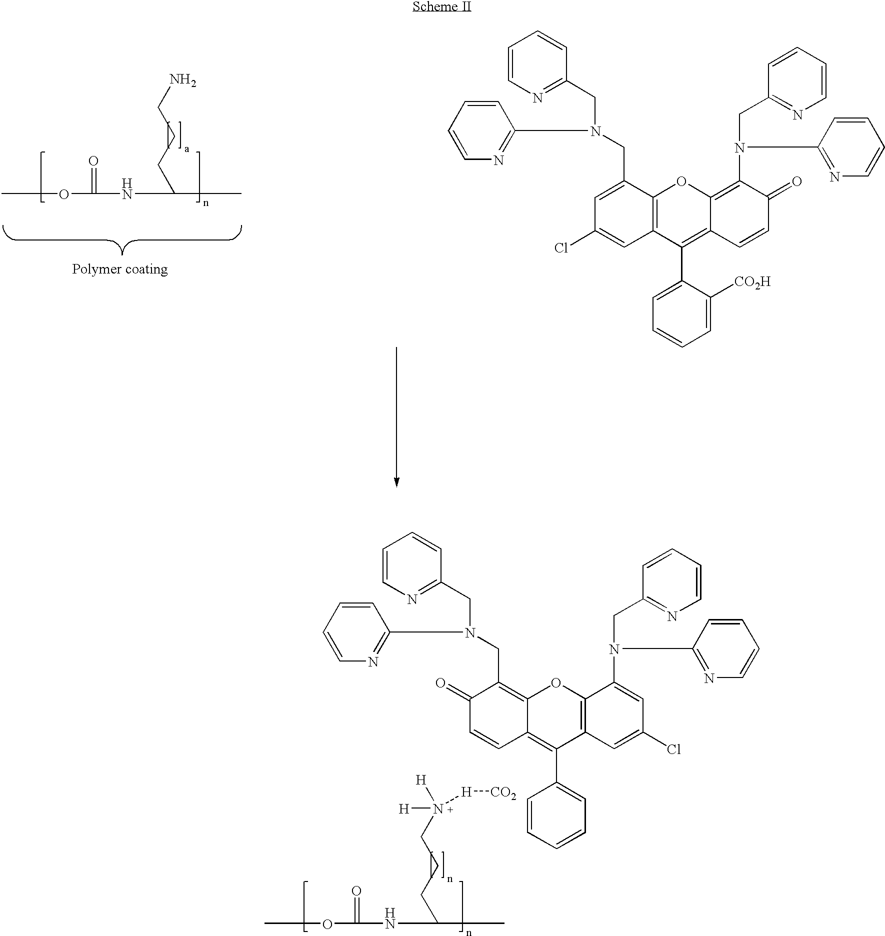

### EXAMPLE 15

Fiber Optic Zinc-Sensing with Zinpyr or TSQ Dyes in Microgel

A proprietary micropolymer was obtained from Gary Kiefer of DOW. Two fluorescence sensors were mixed individually with the micropolymer. One was TFLZN which has a Kd of 10 μM for binding zinc and which is strongly hydrophilic. The other was ZP1, which is more lipophilic, binds zinc with a Kd of 0.5 nM, and is very bright with a quantum yield of 0.9. After mixing, the two solutions were spotted onto acid-cleaned glass slides in spots of 2.5 and 10.0 μL and allowed to cure overnight.

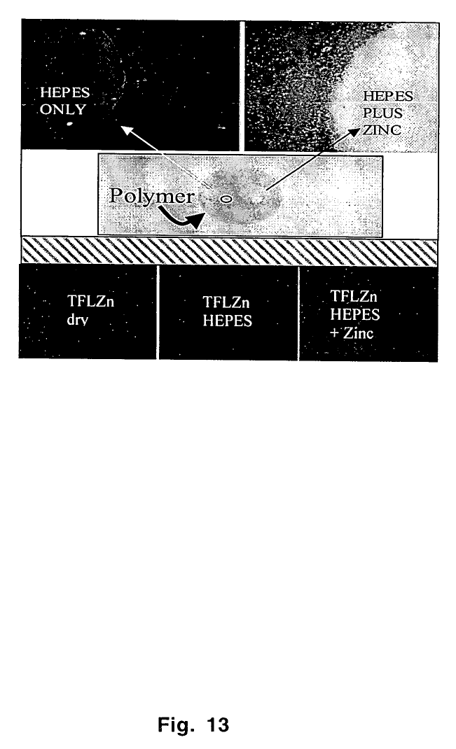

To evaluate the coatings, one microliter of HEPES (50 mM, pH ˜7.4) was put onto one spot on the polymer/probe coatings and a second spot with one microliter of the same HEPES plus 1 microliter of ZnSO4 (final [Zn^{2+]}, 500 μM) was put next to the first spot. Images of the both spots were captured using the exact same camera settings for both spots. FIG. 13 shows the result for the ZP1 test. When the actual fluorescent intensity of the spots was measured using Photoshop, it was observed that the zinc-added spot was from 4-5-fold brighter that the HEPES spot. This is the maximum increase in brightness that ZP1 yields when saturated with zinc at pH 7.4 (58). The change in fluorescence was not timed, but in this microscope-measuring system, the change appeared essentially instantaneously, i.e., within the few seconds it took to put the slide back in place, re-focus the microscope, and capture the second image. This experiment was repeated with four different polymer spots that all turned intensely fluorescent upon adding the zinc. Furthermore, washing the spots with pure HEPES again lowered the fluorescence back to essentially the pre-zinc levels.

These experiments also were repeated with TFLZN-micropolymer spots. Because the exciter for this is UV (360 nm), a higher magnification objective (10×) was needed in order to excite the fluorophore. The increase in fluorescence upon adding zinc to the TFLZN was equally fast and considerably larger than seen with the ZP1. For the TFLZN tests, the shutter speed needed to be decreased by 10-fold from 8 sec for the HEPES spot to 0.8 sec for HEPES plus ZNSO₄ in order to get both images within range for the camera.

A second set of tests were performed with a 3 mm square quartz tube which was coated on the inside with the polymer ZP1 mixture, then spun in radial orientation in a centrifuge at 1200 RPM for 10 min to obtain a thin, potentially monomolecular, coating of the polymer and the ZP1. After overnight curing, the spin-coated quartz tube had a very thin, uniform coating. The quartz tube was placed into the specimen chamber of a SPEX fluorimeter, a HeCAD laser (420 nm) was aimed such that it created a total internal reflection illumination of the quartz walls and the tube was positioned to capture the fluorescent emissions in the monochromator, PMT of the SPEX. Then either a “0” zinc buffer or a 50 μM zinc buffer was flowed through the tube and either spectra or sequential readings at the peak fluorescence of the signal (430 nm) were collected.

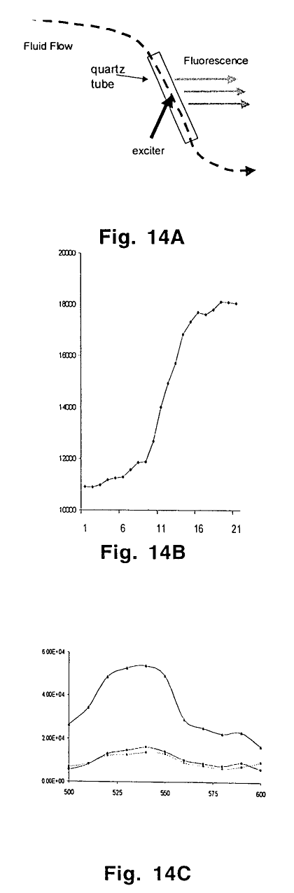

The results (FIGS. 14A-14C) show that the spectra followed the expected changes for −0 zinc, 50 μM zinc, and 0 zinc, although the shape of the spectra were somewhat broader in the flanks that than standard ZP1 spectra (not shown). When the change in intensity over time was followed, during the flow-in of the 50 μM zinc solution, an abrupt rise in fluorescence intensity that peaked within a few seconds was evident. Because it is not known exactly how quickly the zinc-containing fluid actually replaced the low-zinc fluid, the speed of the apparent on response should not be over-interpreted. However, it is noteworthy that the clinical use anticipated for the zintrode fiber is such that a response with a rise time of a few seconds would be of adequate speed for monitoring patients.

### EXAMPLE 16

Cuvette-Based pZn Meter

The pZn meter is a specialized quantitative fluorimeter that is optimized for measuring small volumes of or very low-concentration of about 0.1 nM and up of free zinc, with physiological ranges of about 0.1 nM to about 10 nM, at pHs near 7.4. A prototype comprises a commercially available fluorimeter (Ocean Optics), super bright LED's (470 nm), special order plastic or silica cuvettes (70 μL sample volume), interference filters (Chroma Tech), and hand-machined parts for alignment and cuvette holding.

Alternatively, the fluorimeter may comprise a laser diode (Nichia) such as a 6 mW 475 nm laser diode. The laser diodes monochromaticity, as compared with the 50 nm FWHM of the LED, greatly simplifies the optical filtration necessary. The greater optical power of the laser diode over the LED, within the passband, and the laser diode's choherence simplifies beam control. Furthermore, 405 and 440 nm laser diodes allows the ABDN-CA probes to be used in the pZn meter.

The pZn meter also may incorporate a kinematic cuvette mount to minimize spillage in positioning the sample within the optical path and to minimize sources of optical variation, particularly for non-ratiometric small molecule labels. Additionally, this provides for smaller sample volumes, e.g. 10-50 microliters. It is contemplated that disposable cuvettes designed to handle small volumes and to simplify kinematic mounting are used in the pZn meter.

The prototype can measure 100 pM in 100 μL, or 10⁻¹² moles of Zn:ZP1, i.e., ˜10⁺¹³ molecules. However, considering the properties of the ZP1 dye, for example, extinction coefficient and quantum yield, and the counting efficiency and dark noise of the present fluorimeter, i.e., 87 photons/count where noise=+counts/sec, the best possible performance attainable from the system using 3 LEDS of ˜5 W @ 470 nm can be calculated. This calculation shows that as few as 10 fMoles of Zn:ZP1 theoretically can be counted with an s/n=3. Although it is neither expected to nor needed to attain the theoretical optimum, it is contemplated the pZn meter may be optimized to detect about 1 pM of Zn:ZP1. At 100 μl, this would be a sensitivity of 10¹⁰ molecules. This level of performance means that a user can take as little as 150 μL of sample and measure pZn values as low as 10 (0.1 nM) in a purely affinity binding mode. Because biological fluids have tested pZn's in the 8-9 range, this instrument should serve essentially all users as its useful range is for pZn<10.3, i.e., Zn2+ is greater than about 50 pM.

The range and pH limitations of the Zn meter is to assure accurate performance of the instrument. None of the limitations are problematic because the 50 pM value is low enough that it covers any physiological fluid. In measuring pZn, the overall speciation of the zinc within the fluid being examined must be considered. For example, a complex medium, such as CSF, comprises numerous zinc-binding ligands, each with its own abundance, on rate and off rate. When the abundances and the kinetics of all of the major species are known, one can construct a multi-ligand wquilibrium model and actually predict the concentration of “free” or rapidly-exchangeable zinc, as has been done for blood serum and for sea water. In fact, in these examples, the ligands are a complex buffering system which have two salient properties for the zinc neurobiologist.

The first is the operational level of free zinc, i.e., the pZn, which is the concentration of zinc ions that are available to bind within some specified time interval to a target introduced into the mix. For example, one wants to know what degree of saturation a zinc-receptor with a Ki of 1 nM would reach within seconds or minutes of introducing the fluid onto the cells expressing that receptor.

The second is the zinc-buffering capacity of the fluid, i.e., what abundance of zinc ions the fluid can bind or release under specified conditions. Consider two extreme cases. In the first, a buffered saline is made with HEPES, NaCl and 1 μM of ZnCl₂. The measured pZn of this solution would be 6. For a second solution, the same HEPES and the same NaCl is used, but 50 mM of bicine and 1 mM of ZnCl₂ is added. This second buffer also has a pZn of 6 because the bicine is buffering the Zn²⁺ to 1 μM, based on K. McCall metal buffer calculation programs.

However, although these two solutions have the same pZn, they can demonstrate completely different effects when put on cells or tissue. This is because the two solutions have a 1000-fold different amounts of available zinc. In the first solution, given the right combination of tissue mass and fluid volume, the tissue can bind, and thereby buffer, the zinc down to whatever binding affinity the endogenous ligands have. For example, albumin and thionein, which becomes metallothionein when metalated, will take the free zinc level down to the level dictated by their abundances and their affinities.

Zinc-buffering components may be removed from any complex or biological solutions or fluids by dialyzing through a MW100 dialysis membrane. However, should one want to know the zinc buffering capacity of the biological solutions or other fluids, the solution or fluid first is checked for interfering fluorophores and, finding none, measure the pZn. The buffering capacity of the solution or fluid then can be assessed by adding zinc stoichiometrically and checking for a change in pZn. Alternatively, EDTA may be added to the solution or fluid to remove zinc stoichiometrically, again checking for a change in pZn.

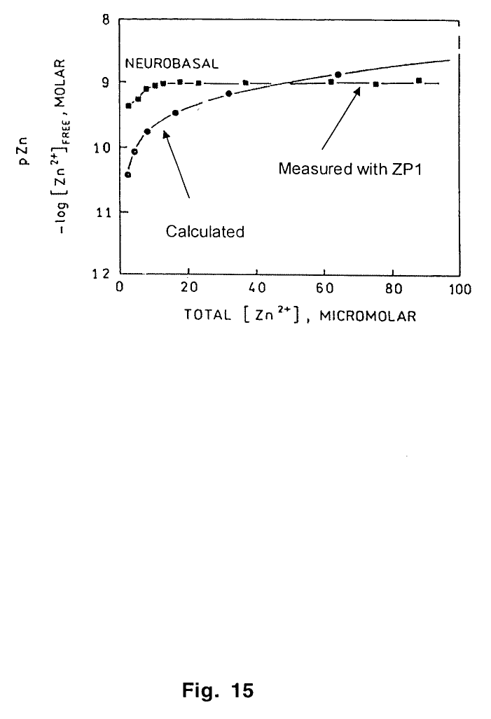

To illustrate the importance of this zinc (ZnCl₂) was added to Neurobasal, a commercial growth medium for neurons. The rise in free zinc, or fall in pZn, is immeasurably small even though a total amount of zinc up to 90 μM was added (FIG. 15). This is because of the zinc-buffering capacity of this particular mix of ingredients. The ingredients from the manufacturer were obtained as proprietary information and we were able to calculate the expected changes in pZn were calculated which confirmed that they were roughly what was observed. This illustrates the importance of pZn measuring capability for those who wish to use free zinc as an independent variable. It MUST be measured.

### EXAMPLE 17

### Calibration of the pZn Meter Using a ZP1 Probe

Using a ZNP1 probe in the pZn meter, a 10 pM concentration of zinc can just be detected and 100-500 pM zinc can be measured as reliably different from zero. It is contemplated that improving optics can increase the limit of detection by about 10-fold to yield accurate measurements of 10 pM. This is based on calculations in an “ideal” optical system which potentially could see as little as 0.1 to 0.01 pM of Zn:ZP1 in solution in a 100 μl cuvette.

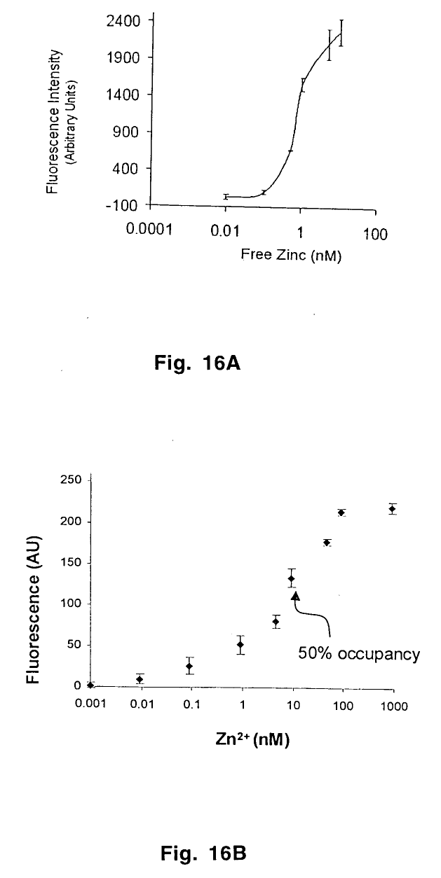

In calibrating the pZn meter using a metal buffer to control free zinc (FIG. 16A), the [ZP1] is <<than the effective [Zn2+ free] because the metal buffer, 50 μM bicine, can supply a large total number of Zn ions, while keeping the concentration of zinc ions fixed. Thus, in this calibration, the fluorescence intensity midpoint is approximately equal to the KD, 0.5 nM where [ZP1]=100 nM. In a second calibration, a ZP1 concentration of 20 nM of zinc, as ZnSO₄, is used (FIG. 16B). Because no metal buffer is used and because the ZP1 concentration is much higher than the zinc concentration, this is a stoichiometric or “percent occupancy” titiration in which the fluorescence intensity is simply proportional to the molar ratio, [Zn]:[ZP1}.

### EXAMPLE 18

Clean-Up Cuvette

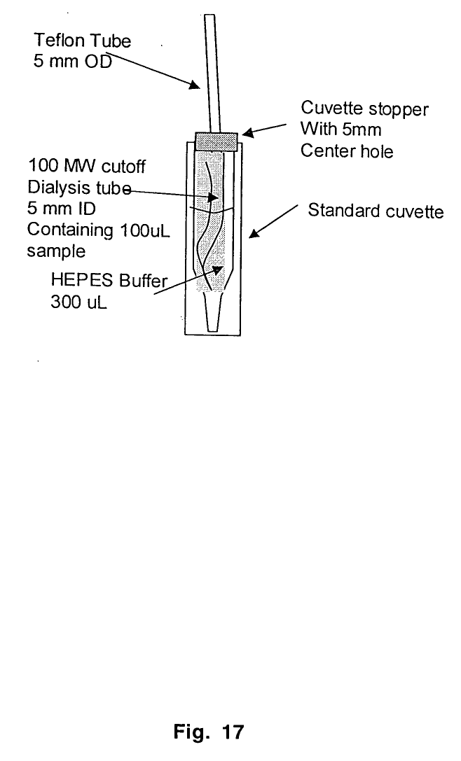

To measure pZn in biological solutions it is intended that measurements be take only after cleaning up the sample by dialysis through the MW100 cutoff dialysis membrane to remove essentially all organics. The pZn meter may comprise this clean-up system to reduce greatly the chance that any organic zinc-buffering ligands in the dialysate are present in the sample in which the pZn is measured. The key components of the “clean up” kit are the MW 100 membrane (Spectrum Laboratories), a cuvette, a cuvette stopper and a short piece of ˜5 mm OD TFE Teflon (Small Parts) (FIG. 17). The bottom of the dialysis tube may be sealed by, but not limited to, crimping under high pressure or a bonding method, such as gluing or heat welding. The components are free of or have been cleaned of adventitious zinc.

### EXAMPLE 19

Measuring pZn Using Total Internal Reflection Fluorescence (TIRF)

TIRF permits extremely small sample volumes to be assayed and suppresses optical background. The fluorescent dye is bonded to the inner surface of the cuvette described in Example 15. Total internal reflection illuminates the walls of the cuvette, so that fluorescence interrogation will take place only within the few hundred nanometers of fluid closest to the cuvette wall, i.e., within the illumination of evanescent wall. The proximity-dependent, evanescent wave method eliminates the need to clean up samples by dialysis, since only molecules that are tethered close to the wall, as are the fluorescent probe and any “captured zinc” will be illuminated for any period of time by the evanescent wave. This requires illuminating for maximum excitation energy, e.g., 478 nm, with the minimum crossover into the emission spectrum.

### EXAMPLE 20

Components of a Multitrode System

The existing Optex system comprises: (1) a real-time intra-arterial catheter sensor or tritode; (2) a 180 degree bend at the fiber tip of each fiber optic channel (FIG. 10), improving optical performance; (3) an arterial introducer or cannula, which provides arterial access housing for the multi-channel zintrode; and (4) the monitoring hardware. The existing Optex tritode has three separate fiber optic channels measuring pH, pO2 and pCO2. A fourth channel represented by the carbonic anhydrase-based zinc biosensor forms a multitrode.

Each tritode is guided into the A-line catheter and then locked into position via a lever lock ‘Y’ fitting, which provides to the physician access for blood sample collection and for pressure monitoring. The zinc-sensing zintrode is placed in a calibration cuvette containing a calibration fluid during product packaging. The base unit contains a high-resolution graphic display screen, and a bank of user interface soft keys for control of monitoring functions. The sensor interface unit includes a light source, detection components and electronic data amplification elements.

The following references are cited herein:
1. Prasad, A. S., Nutrition, 17(7-8), 685-7 (2001).
2. Berg, J. M., Ann Rev Biophys Biophys Chem, 19, 405-21 (1990).
3. Vallee, B. L. and Auld, D. S., EXS, 73, 259-77 (1995).
4. Frederickson, C. J., Int Rev Neurobiol, 31, 145-238 (1989).
5. Wallwork, J. C., Prog Food Nutr Sci, 11 (2), 203-47 (1987).
6. Howell et al., Nature, 308, 736-8 (1984).
7. Frederickson, C. J. and Bush, A. I., Biometals, 14(3-4), 353-66 (2001).
8. Weiss et al., Sensi. Trends Neurosci, 23, 365-71 (2000).
9. Choi, D. W. and Koh, J. Y., Ann Rev Neurosci, 21, 347-75 (1998).
10. Baranano et al., Trends Neurosci, 24(2), 99-106 (2001).
11. Palmiter et al., EMBO J, 15, 1784-91 (1996a).
12. Palmiter et al., Proc Nat Acad Sci, 93, 14934-14939 (1996b).
13. Lu et al., Synapse, 38, 187-97 (2000).
14. Li et al., J Neurophysiol, 86, 2597-2604 (2001 a).
15. Long, Y. Y. and Frederickson, C. J., Neuroreports, 5, 2026-8 (1994).
16. Mandava et al., Brain Res, 608, 115-22 (1993).
17. Slomianka et al. Anat Embryol (Berl), 195(2), 165-74(1997).
18. Long et al., Soc Neurosci Abstr, 24, 1727 (1998).
19. Beaulieu et al., Neuroreport, 3, 861-4 (1992).
20. Cole et al., Proc Nat Acad Sci, 96(4), 1716-21 (1999).
21. Li et al., J Neurosci, 21(20), 8015-25 (2001b).
22. Vogt et al., Neuron, 26(1), 187-96 (2000).
23. Bush et al., Science, 265, 1464-67 (1994).
24. Bush et al., Science, 268, 1921-23 (1995).
25. Mitchell et al., Brain Res, 506, 327-30 (1990).
26. Frederickson et al., Brain Res, 480, 317-21 (1989).
27. Tonder et al., Neurosci Lett, 109, 247-52 (1990).
28. Sorenson et al., Brain Res, 812, 265-9 (1998).
29. Suh et al., Brain Res, 852, 268-73 (2000).
30. Prough et al., J Neurotrauma (submitted) (2002).
31. Cuajungco, M. P. and Lees, G. J., Brain Res, 799(1), 118-29 (1998).
32. Morton et al., Epilepsia, 31, 139-44(1990).
33. Lees et al., Brain Res, 799, 108-117 (1998).
34. Long et al., Soc Neurosci Abstr, 24, 1727 (1998).
35. Frederickson et al., J Histochem Cytochem, 50(12), 1659-62 (2002a).
36. Mitchell, C. L. and Barnes, M. L., Physiol Behav, 53(1), 45-9 (1993).
37. Lin et al., J Neurophysiol, 85(3), 1185-96 (2001).
38. Snider et al., Neuro Sci, 100(3), 651-61 (2000).
39. Aizenman et al., J Neurochem., 75(5), 1878-88 (2000).
40. Frederickson et al., Neuroscience, 115(2), 471-4 (2002b).
41. Cherny et al., Neuron, 30(3), 665-76 (2001).
42. Truong-Tran et al., Biometals. 14(3-4), 315-30 (2001).
43. Thompson et al. J Neurosci Meth, 118, 63-75 (2002).
44. Thompson et al., J Neurosci Methods, 96, 35-45 (2000).
45. Krebs, J. F. and Fierke, C. A., J Biol Chem, 268, 948-54 (1993).
46. Alexander et al., Biochemistry, 32, 1510-1518 (1993).
47. Sun et al., Synthesis of fluorinated fluoresceins. J. Org. Chem. 62, 6469-6475 (1997).
48. Munkholm et al. J Am Chem Soc 112, 2608-2612 (1990).
49. Burdette et al, J Am Chem Soc, 125(7), 1778-1787 (2003).
50. Thompson, et al., Soc Neurosci Abs. (2001).
51. Fierke, C. A. and Thompson, R. B., Biometals, 14(3-4), 205-22 (2001).
52. Hanahan, J., J Mol Biol, 166(4), 557-80 (1983).
53. Woodroofe, C. C. and Lippard, S. J., J. Am. Chem. Soc. 125, 11458-11459 (2003).
54. Walkup, et al., J. Am. Chem. Soc. 122, 5644-5645 (2000).
55. Schaap et al., J. Am. Chem. Soc. 97, 3741-3745 (1975).
56. Webber, S. E. and Hargreaves, J. S., Can. J. Chem. 63, 1320-1327 (1985).
57. Manku et al., J. Comb. Chem. 5, 379-391 (2003).
58. S. C. Burdette et a.l, J Am Chem Soc, 125(7), 3511 (2003).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A zintrode to measure free zinc levels in a solution, comprising:
a chelator to chelate free zinc ions;
a fluorophore;
an optical fiber comprising an optical tip; and
means of entrapping said chelator and said fluorophore proximate to the optical tip within the zintrode.

2. The zintrode of claim 1, wherein said chelator is a wild-type apoenzyme or genetically engineered mutant thereof.

3. The zintrode of claim 2, wherein said apoenzyme is wild-type apo-carbonic anhydrase, a S166C mutant, a H36C mutant or a E117A mutant.

4. The zintrode of claim 2, wherein said fluorophore is DPSA, DNSA, Butyl-DPSA or polyABDN.

5. The zintrode of claim 4, wherein said fluorophore is covalently tethered to controlled-pore glass beads or to a protein or physically bound to a polyethylene thread.

6. The zintrode of claim 2, wherein said means of entrapping is a microdialysis chamber comprising a semi-permeable membrane positioned over the tip of said optical fiber and attached thereto, said apoenzyme and said fluorophore entrapped therein.

7. The zintrode of claim 2, wherein said means of entrapping is a V-notch cut into a U-bend at the tip of said optical fiber with a semi-permeable microdialysis membrane positioned over the V-notch and attached thereto, said apoenzyme and said fluorophore entrapped therein.

8. The zintrode of claim 2, wherein said means of entrapping is a water-soluble polymer matrix polymerized onto the tip of said optical fiber, said fluorophore conjugated to said apoenzyme and embedded therein.

9. The zintrode of claim 8, wherein said water-soluble polymer matrix is a sol-gel, a hydrogel or a microgel.

10. The zintrode of claim 1, wherein said zinc chelator is said fluorophore.

11. The zintrode of claim 10, wherein said zinc-chelating fluorophore is a zinc-chelating fluorescent dye.

12. The zintrode of claim 11, wherein said zinc-chelating fluorescent dye is a zinpyr dye, TSQ or a congener thereof or a rhodafluor dye.

13. The zintrode of claim 12, wherein said zinpyr dye is 9-(O-carboxyphenyl)-2,7-dichloro-4,5-bis[bis(2-pyridylmethyl)-aminomethyl]-6-hydroxy-3-xanthanone, 9-(O-carboxyphenyl)-4,5-bis[bis(2-pyridylmethyl)-aminomethyl]-6-hydroxy-3-xanthanone, 9-(O-carboxyphenyl)-2-chloro-5-[2-{bis(2-pyridylmethyl)aminomethyl}-N-methylaniline]-6-hydroxy-3-xanthanone, ZP-3 or ZPN.

14. The zintrode of claim 12, wherein said congener of TSQ is TFLZN.

15. The zintrode of claim 12, wherein said rhodafluor dye is (1-[9′-(o-carboxyphenyl)-6′-amino-2′-chloro-3′-xanthanone]-4,10-(diethyl)-7-(2-pyridylmethyl)-1,4,7,10-tetraazacyclododecane.

16. The zintrode of claim 10, wherein said means of entrapping is a covalent bond between said zinc-chelating fluorophore and the optical tip.

17. The zintrode of claim 10, wherein said means of entrapping is a water-soluble polymer matrix polymerized onto the tip of said optical fiber, said zinc-chelating fluorophore embedded therein.

18. The zintrode of claim 10, wherein said water-soluble polymer matrix is a sol-gel, a hydrogel or a microgel.

19. The zintrode of claim 10, wherein said means of entrapping is a microdialysis chamber comprising a capillary tube containing said zinc-chelating fluorophore at one end and having said optical tip inserted therein and a semi-permeable membrane positioned over the capillary tube and attached thereto, said zinc-chelating fluorophore entrapped therein.

20. The zintrode of claim 10, wherein said means of entrapping is a capillary tube containing said zinc-chelating fluorophore at one end in a solution of a highly viscous low polarity liquid and having said optical tip inserted therein, said zinc-chelating fluorophore entrapped therein.

21. The zintrode of claim 20, wherein said liquid is a high molecular weight alcohol, a high molecular weight fatty acid or a silicone oil.

22. The zintrode of claim 20, wherein the end of said capillary tube is narrowed to about 50 microns.

23. The zintrode of claim 1, wherein said solution is a growth media comprising fetal calf serum, cerebral spinal fluid, blood, seminal serum, saliva, tears, urine, or synthetic salt solutions.

24. A kit comprising:
the zintrode of claim 1; and
a zinc calibration solution.

25. A method of real-time buffering of zinc ion levels in vivo in brain tissue to treat a pathological condition characterized by abnormal levels of zinc, comprising the steps of:
a) intracranially implanting the zintrode of claim 1 into the brain tissue;
b) measuring the level of zinc ions in the brain tissue with said zintrode;
c) administering a pharmacologically effective dose of an agent to remove an amount of zinc ions sufficient to maintain the level of remaining zinc ions within a physiologically acceptable range;
d) continuously monitoring the zinc ion level in step b); and
e) repeating step c) if the monitored level of zinc ions increases above the maintenance level thereby buffering the zinc ion levels in brain tissue in real-time to treat said pathological condition.

26. The method of claim 25, further comprising:
implanting an intra-arterial tritrode comprising fiber optic channels to measure one of blood pH, pO₂ or pCO₂;
measuring a level of blood pH, pO₂ or pCO₂; and
monitoring said blood pH, pO₂ or pCO₂ levels during buffering of zinc ion levels.

27. The method of claim 25, wherein measuring the level of zinc ions in brain tissue comprises:
diffusing zinc ions into said zintrode for a period of time;
delivering an excitation maximum wavelength to a fluorophore in said zintrode via an optical tip comprising said zintrode;
measuring emission maximum wavelengths, said fluorophore emitting one emission maximum wavelength or two different emission maximum wavelengths; and
correlating the level of zinc ions with said one emission maximum wavelength or ratiometrically from said two different emission maximum wavelengths.

28. The method of claim 27, wherein said fluorophore complexes with a wildtype or mutated apocarbonic anhydrase and holocarbonic anhydrase, each complex emitting one of said two different emission maximum wavelengths.

29. The method of claim 28, wherein said mutant apo- or holocarbonic anhydrase is a S166C mutant, a H36C mutant or a E117A mutant.

30. The method of claim 28, wherein said fluorophore is DPSA, DNSA, Butyl-DPSA or polyABDN.

31. The method of claim 27, wherein said fluorophore complexes with said zinc ions, said zinc ion level determined from the emission maximum wavelength of said fluorophore-zinc complex.

32. The method of claim 31, wherein said fluorophore is 9-(O-carboxyphenyl)-2,7-dichloro-4,5-bis[bis(2-pyridylmethyl)-aminomethyl]-6-hydroxy-3-xanthanone, 9-(O-carboxyphenyl)-4,5-bis[bis(2-pyridylmethyl)-aminomethyl]-6-hydroxy-3-xanthanone, 9-(O-carboxyphenyl)-2-chloro-5-[2-{bis(2-pyridylmethyl)aminomethyl}-N-methylaniline]-6-hydroxy-3-xanthanone, ZP3, ZPN, TSQ, or TFLZN.

33. The method of claim 27, wherein said fluorophore complexes with said zinc ions, said zinc ion level determined ratiometrically from the emission maximum wavelengths of said fluorophore and said fluorophore-zinc complex.

34. The zintrode of claim 33, wherein said fluorophore is (1-[9′-(O-carboxyphenyl)-6′-amino-2′-chloro-3′-xanthanone]-4,10-(diethyl)-7-(2-pyridylmethyl)-1,4,7,10-tetraazacyclododecane.

35. The method of claim 25, wherein said zinc ion levels are continuously monitored via a base unit comprising:
a high resolution graphic display screen; and
a sensor interface unit comprising:
a light source;
photodiode detectors, optionally with bandpass filters; and
electronic data amplification elements.

36. The method of claim 25, wherein said physiologically acceptable range of zinc ions is about 0.1 nM to about 20 nM.

37. The method of claim 36, wherein said physiologically acceptable range of zinc ions is about 0.5 nM to about 5 nM.

38. The method of claim 37, wherein said physiologically acceptable range of zinc ions is about 1 nM.

39. The method of claim 25, wherein said agent to remove zinc ions from said brain tissue is a zinc-release blocker, a zinc chelator or a zinc channel blocker.

40. The method of claim 39, wherein said zinc chelator is clioquinol.

41. The method of claim 25, wherein said pathological condition is an excitotoxic neural injury, epilectic seizure, ischemia, Alzheimer's disease or traumatic brain injury.

42. An implantable multitrode to measure intracranial physiological parameters in brain tissue in an individual, comprising:
an intra-arterial tritrode comprising fiber optic channels to monitor one of blood pH, pO₂ or pCO₂ parameters;
an implantable zintrode to measure free zinc ion levels in the brain tissue, comprising:
a zinc-chelating fluorophore;
an optical fiber having an optical tip; and
means of attaching said zinc-chelating fluorophore to the optical tip; and
means of electronically monitoring the multitrode.

43. The multitrode of claim 42, wherein said zinc-chelating fluorophore is a zinc-chelating fluorescent dye.

44. The multitrode of claim 43, wherein said zinc-chelating fluorescent dye is a zinpyr dye or TSQ or a congener thereof.

45. The multitrode of claim 44, wherein said zinpyr dye is 9-(O-carboxyphenyl)-2,7-dichloro-4,5-bis[bis(2-pyridylmethyl)-aminomethyl]-6-hydroxy-3-xanthanone, 9-(O-carboxyphenyl)-4,5-bis[bis(2-pyridylmethyl)-aminomethyl]-6-hydroxy-3-xanthanone or 9-(O-carboxyphenyl)-2-chloro-5-[2-{bis(2-pyridylmethyl) aminomethyl}-N-methylaniline]-6-hydroxy-3-xanthanone, ZP-3 or ZPN.

46. The multitrode of claim 44, wherein said congener of TSQ is TFLZN.

47. The multitrode of claim 42, wherein said means of attaching is a covalent bond between said zinc-chelating fluorophore and the optical tip.

48. The multitrode of claim 42, wherein said means of attaching is a water-soluble polymer matrix polymerized onto the tip of said optical fiber, said zinc-chelating fluorophore embedded therein.

49. The multitrode of claim 42, wherein said water-soluble polymer matrix is a sol-gel, a hydrogel or a microgel.

50. The multitrode of claim 42, wherein said means of electronically monitoring said multitrode is a base unit comprising:
a high resolution graphic display screen; and
a sensor interface unit comprising:
a light source;
detection components for said tritrode;
photodiode detectors, optionally with bandpass filters, for said zintrode; and
electronic data amplification elements.

51. A method of real-time buffering of zinc ion levels in vivo in brain tissue to treat a pathological condition characterized by abnormal levels of zinc, comprising the steps of:
a) implanting the multitrode of claim 21 into the brain tissue;
b) continuously monitoring blood pH, pO2 and pCO2 via the tritrode comprising said multitrode;
c) measuring the level of zinc ions in the brain tissue with the zintrode comprising said multitrode;
d) administering a pharmacologically effective dose of an agent to remove an amount of zinc ions sufficient to maintain the level of remaining zinc ions within a physiologically acceptable range;
e) continuously monitoring the zinc ion level in step c); and
f) repeating step d) if the monitored level of zinc ions increases above the maintenance level thereby buffering the zinc ion levels in brain tissue in real-time to treat said pathological condition.

52. The method of claim 51, wherein measuring the level of zinc ions in brain tissue comprises:
diffusing zinc ions into said zintrode for a period of time;
delivering an excitation maximum wavelength to said zinc-chelating fluorophore in said zintrode via an optical tip comprising said zintrode;
measuring an emission maximum wavelength emitted by said zinc-chelating fluorophore; and
correlating the level of zinc ions with said emission maximum wavelength.

53. The method of claim 51, wherein said physiologically acceptable range of zinc ions is about 0.1 nM to about 20 nM.

54. The method of claim 53, wherein said physiologically acceptable range of zinc ions is about 0.5 nM to about 5 nM.

55. The method of claim 54, wherein said physiologically acceptable range of zinc ions is about 1 nM.

56. The method of claim 51, wherein said agent to remove zinc ions from said brain tissue is a zinc-release blocker, a zinc chelator or a zinc channel blocker.

57. The method of claim 56, wherein said zinc chelator is clioquinol.

58. The method of claim 51, wherein said tritrode is implanted via an arterial introducer or via a cannula.

59. The method of claim 51, wherein said zintrode is implanted via a cannula.

60. The method of claim 51, wherein said pathological condition is an excitotoxic neural injury, epilectic seizure, ischemia, Alzheimer's disease or traumatic brain injury.

61. A system to measure pZn in a solution, comprising:
a zintrode comprising;
a zinc-chelating fluorophore;
an optical fiber comprising an optical tip; and
means of entrapping said zinc-chelating fluorophore proximate to the optical tip within the zintrode;
means for holding the solution; and
optical components to measure fluorescence.

62. The system of claim 61, further comprising:
a means for removing zinc-binding ligands from said solution prior to measuring pZn.

63. The system of claim 62, wherein said removing means comprises:
a 100 MW dialysis tube;
a cuvette containing said dialysis tube; and
a buffer.

64. The system of claim 61, wherein said zinc-chelating fluorophore is a zinc-chelating fluorescent dye.

65. The system of claim 64, wherein said zinc-chelating fluorescent dye is a zinpyr dye or TSQ or a congener thereof.

66. The system of claim 65, wherein said zinpyr dye is 9-(O-carboxyphenyl)-2,7-dichloro-4,5-bis[bis(2-pyridylmethyl)-aminomethyl]-6-hydroxy-3-xanthanone, 9-(O-carboxyphenyl)-4,5-bis[bis(2-pyridylmethyl)-aminomethyl]-6-hydroxy-3-xanthanone, 9-(O-carboxyphenyl)-2-chloro-5-[2-{bis(2-pyridylmethyl)aminomethyl}-N-methylaniline]-6-hydroxy-3-xanthanone, ZP-3 or ZPN.

67. The system of claim 65, wherein said congener of TSQ is TFLZN.

68. The system of claim 61, wherein said means of entrapping is a covalent bond between said zinc-chelating fluorophore and the optical tip.

69. The system of claim 61, wherein said means of entrapping is a water-soluble polymer matrix, said zinc-chelating fluorophore embedded therein.

70. The system of claim 69, wherein said water soluble polymer matrix is polymerized onto the optical tip of the optical fiber.

71. The system of claim 69, wherein said water soluble polymer matrix is polymerized onto the inner surfaces of a cuvette, said cuvette comprising the means for holding the solution.

72. The system of claim 69, wherein said water-soluble polymer matrix is a sol-gel, a hydrogel or a microgel.

73. The system of claim 61, wherein said means for holding the solution is a cuvette.

74. The system of claim 61, wherein said solution is a growth media comprising fetal calf serum, cerebral spinal fluid, blood, seminal serum, saliva, tears, urine, or synthetic salt solutions.

75. A method of measuring pZn in a solution, comprising:
a) contacting the solution with a zintrode comprising the system of claim 61;
b) diffusing free zinc ions in the solution into said zintrode for a period of time;
c) delivering an excitation maximum wavelength to said zinc-chelating fluorophore in said zintrode via the optical tip comprising said zintrode;
d) measuring an emission maximum wavelength emitted by said zinc-chelating fluorophore via the optical components comprising the system; and
e) correlating the concentration of free zinc ions with said emission maximum wavelength thereby measuring the pZn of the solution.

76. The method of claim 75, further comprising prior to step a):
placing the solution into a 100 MW dialysis tube contained within a cuvette; and
dialyzing said solution against a buffer to remove zinc-binding ligands.

77. The method of claim 75, wherein the concentration of free zinc ions is greater than about 50 picomolar.

78. The method of claim 75, wherein a measurable pZn is less than about 10.3.
